(19) United States
(12) Patent Application Publication
(10) Pub. No.: US 2006/0018786 A1
(43) Pub. Date: Jan. 26, 2006

(54) Multi-compartment container for use in producing an aroma

(75) Inventors: Steven Tolman, Ogden, UT (US); Lisa Tolman, Ogden, UT (US)

Correspondence Address: KARL R CANNON, PO BOX 1909, SANDY, UT, 84091, US

(73) Assignee: JC Candle Company, Inc.

(21) Appl. No.: 10/895,808

(22) Filed: Jul. 20, 2004

(51) Int. Cl. A61L 9/03; A61L 9/02

(52) U.S. Cl. 422/5; 422/125

(57) ABSTRACT

A multi-compartment container for receiving a fragrance producing medium, such as scented wax, for use with a warmer to produce a pleasant aroma. The container may have multiple parts that may be moveable with respect to each other and configured to receive a different scented fragrance producing medium, such that custom blended aromas may be formulated. The container may be specifically configured to be received in a recess in the warmer such that the container may be held in a stable condition. The container may be formed in various different shapes and configurations, and may be provided with various different accessories. Accordingly, the container and warmer may be versatile for use with various decorative themes and occasions.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND

1. The Field of the Invention.

The present disclosure relates generally to devices and methods for use in producing an aroma, and more particularly, but not necessarily entirely, to a multi-compartment container that receives a fragrance producing medium, such as scented candles, for use on a warmer device.

2. Description of Related Art

Scented candles are known in the art for producing a pleasant aroma. The scented candles may have various different fragrances which may be enhanced as the candles are burned. Heat generated by burning a wick may cause additional fragrance to be released from the candle wax. A drawback of burning the scented candles is that burning the candles consumes the candles and produces smoke that may tarnish walls and furnishings. Also, the smoke produced may cause irritation to the eyes and lungs of those who are close enough to the scented candles to enjoy the fragrances they produce. Moreover, flame generated while burning a wick may cause a safety hazard in that unwanted fire or burning injury may result.

Candle warmers used with scented candles are increasing in popularity as a clean safe way to produce pleasant aromas without the use of a flame. The warmers may include a heating surface, such as an electrically heated plate. Scented candles may be placed on the heating surface so as to warm the candle and increase the amount of fragrance released. Since a wick is not burned with a flame, the emission of harmful and irritating substances can be eliminated. Also, the candle is not consumed such that the candle may be available for use for a longer period of time. Moreover, safety is enhanced since the risk of fire or burning may be reduced.

The scented candles known for use with candle warmers are configured to have a particular scent for each candle. Accordingly, the fragrances produced by a candle are limited to the particular scent of the candle, and custom blended aromas are difficult to achieve. Also, the prior art candle and warmer systems are configured such that it is difficult to adjust the strength of the fragrance emitted. Moreover, the presently known scented candles are not placed in containers or bowls that are specifically configured for use with warmers. Thus, the candles may not be supported in a stable manner on the warmer, or excessive areas of the heated portion of the warmer may be exposed, thereby reducing the efficiency of the heat transferred by the warmers. Additionally, the known warmers and candles are less versatile since containers having multiple shapes may not be compatible with a particular warmer.

The prior art is thus characterized by several disadvantages that are addressed by the present disclosure. The present disclosure minimizes, and in some aspects eliminates, the above-mentioned failures, and other problems, by utilizing the methods and structural features described herein.

The features and advantages of the disclosure will be set forth in the description which follows, and in part will be apparent from the description, or may be learned by the practice of the disclosure without undue experimentation. The features and advantages of the disclosure may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a perspective view of an apparatus for producing an aroma including a container having a plurality of compartments on a warmer;

FIG. 2a is a perspective view of a portion of the container of FIG. 1, the portion comprising one third of the container such that the container may form a three piece circular set;

FIG. 2b is an end view of the portion of the container of FIG. 2a;

FIG. 2c is a side view of the portion of the container of FIG. 2a;

FIG. 2d is a top view of the portion of the container of FIG. 2a;

FIG. 3a is a perspective view of an alternative embodiment portion of a container in which sidewalls are convexly curved;

FIG. 3b is an end view of the portion of the container of FIG. 3a;

FIG. 3c is a side view of the portion of the container of FIG. 3a;

FIG. 3d is a top view of the portion of the container of FIG. 3a;

FIG. 4a is a perspective view of an alternative embodiment portion of a container in which sidewalls are concavely curved;

FIG. 4b is an end view of the portion of the container of FIG. 4a;

FIG. 4c is a side view of the portion of the container of FIG. 4a;

FIG. 4d is a top view of the portion of the container of FIG. 4a;

FIG. 5a is a perspective view of an alternative embodiment portion of a container in which the portion is one fourth of the container such that the container forms a four piece circular set;

FIG. 5b is an end view of the portion of the container of FIG. 5a;

FIG. 5c is a side view of the portion of the container of FIG. 5a;

FIG. 5d is a top view of the portion of the container of FIG. 5a;

FIG. 6a is a perspective view of an alternative embodiment portion of a container in which the portion is one half of the container such that the container forms a two piece circular set;

FIG. 6b is an end view of the portion of the container of FIG. 6a;

FIG. 6c is a side view of the portion of the container of FIG. 6a;

FIG. 6d is a top view of the portion of the container of FIG. 6a;

FIG. 7a is a perspective view of an alternative embodiment portion of a container, in which the portion is heart shaped;

FIG. 7b is a top view of a container having four heart shaped portions;

FIG. 8a is a top view of an alternative embodiment of a container, wherein the container is a multi compartment, multi piece container having an upper square shape and a round lower shape;

FIG. 8b is a top view of an alternative embodiment of a container, wherein the container is a multi compartment, multi piece container having an upper round shape and a round lower shape;

FIG. 8c is a top view of an alternative embodiment of a container, wherein the container is a multi compartment, multi piece container having an upper clover shape and a round lower shape;

FIG. 8d is a top view of an alternative embodiment of a container, wherein the container is a multi compartment, multi piece container having an upper fruit shape and a round lower shape;

FIG. 8e is a top view of an alternative embodiment of a container, wherein the container is a multi compartment, multi piece container having an upper heart shape and a round lower shape;

FIG. 8f is a top view of an alternative embodiment of a container, wherein the container is a multi compartment, multi piece container having an upper tree shape and a round lower shape;

FIG. 8g is a top view of an alternative embodiment of a container, wherein the container is a multi compartment, multi piece container having an upper star shape and a round lower shape;

FIG. 8h is a top view of an alternative embodiment of a container, wherein the container is a multi compartment, multi piece container having an upper triple heart shape and a round lower shape;

FIG. 8i is a top view of an alternative embodiment of a container, wherein the container is a multi compartment, multi piece container having an upper diamond shape and a round lower shape;

FIG. 8j is a top view of an alternative embodiment of a container, wherein the container is a multi compartment, multi piece container having an upper animal shape and a round lower shape;

FIG. 8k is a top view of an alternative embodiment of a container, wherein the container is a multi compartment, multi piece container having an upper flower shape and a round lower shape;

FIG. 8l is a top view of an alternative embodiment of a container, wherein the container is a multi compartment, multi piece container having an upper leaf shape and a round lower shape;

FIG. 9 is a side cross sectional view of an alternative embodiment of a portion of a container;

FIG. 10 is a side break-away view of an outer edge of an alternative embodiment of a portion of a container;

FIG. 11a is an exploded perspective view of a cover for a warmer;

FIG. 11b is a side view of the cover of FIG. 11a, with a cross-sectional view of a warmer;

FIG. 11c is a top view of the cover of FIG. 11a;

FIG. 12a is an exploded perspective view of a further embodiment cover and a multi compartment, single piece container;

FIG. 12b is a side cross-sectional view of the cover of FIG. 12a;

FIG. 12c is a top view of the cover of FIG. 12a with the container removed;

FIG. 13a is a perspective view of an additional embodiment cover having an integral multi-compartment container;

FIG. 13b is a side view of the cover of FIG. 13a;

FIG. 13c is a top view of the cover of FIG. 13a;

FIG. 14a is a top view of an additional embodiment cover having a frusto-pyramidal shape with a multi compartment container;

FIG. 14b is an exploded side view of the cover of FIG. 14a, with a lid;

FIG. 14c is a top view of the cover and lid of FIG. 14b;

FIG. 14d is a perspective view of the cover of FIG. 14a;

FIG. 15a is an exploded perspective view of a further embodiment cover;

FIG. 15b is a side view of the cover of FIG. 15a, with a cross-sectional view of a warmer;

FIG. 15c is a top view of the cover of FIG. 15a;

FIG. 16a is an exploded view of an additional embodiment container, lid and warmer;

FIG. 16b is a top view of an alternative embodiment lid;

FIG. 16c is a top view of an additional embodiment lid;

FIG. 17 is an exploded perspective view of an alternative embodiment multi compartment, multi piece container, and bowl;

FIG. 18a is a perspective view of an alternative embodiment multi compartment, multi piece container and warmer, having a square shape;

FIG. 18b is a perspective view of an additional embodiment multi compartment, multi piece container and warmer, having a triangular shape;

FIG. 18c is a perspective view of a further embodiment multi compartment, multi piece container and warmer, having an alternative curved shape;

FIG. 19 is a perspective view of an additional embodiment container, wherein the container is a multi compartment, single piece container;

FIG. 20 is a top view of the container of FIG. 19;

FIG. 21a is a top view of an alternative embodiment multi compartment, single piece container;

FIG. 21b is a top view of an additional embodiment multi compartment, single piece container;

FIG. 21c is a top view of a further alternative embodiment multi compartment, single piece container;

FIG. 21d is a top view of an additional embodiment multi compartment, single piece container;

FIG. 21e is a top view of a further alternative embodiment multi compartment, single piece container;

FIG. 21f is a top view of another alternative embodiment multi compartment, single piece container;

FIG. 21g is a top view of a further alternative embodiment multi compartment, single piece container;

FIG. 21h is a top view of a further alternative embodiment multi compartment, single piece container;

FIG. 21i is a top view of a further alternative embodiment multi compartment, single piece container;

FIG. 21j is a top view of a further alternative embodiment multi compartment, single piece container;

FIG. 21k is a top view of a further alternative embodiment multi compartment, single piece container;

FIG. 21l is a top view of a further alternative embodiment multi compartment, single piece container;

FIG. 22 is an exploded perspective view of an alternative embodiment multi compartment, single piece container insert, bowl and warmer;

FIG. 23 is a perspective view of an additional embodiment container and warmer in which the container and warmer are one-piece;

FIG. 24a is an exploded perspective view of an alternative embodiment container with a modular divider;

FIG. 24b is an exploded perspective view of a further embodiment container with a modular divider;

FIG. 25a is a perspective view of a further embodiment container having concentric compartments with different heights;

FIG. 25b is a perspective view of an additional embodiment container with concentric compartments having the same height;

FIG. 25c is an exploded perspective view of the embodiment of the container of FIG. 25b;

FIG. 26 is an exploded perspective view of an additional embodiment container with concentric compartments;

FIG. 27 is a perspective view of an additional embodiment container having multiple compartments of non-uniform shapes and heights;

FIG. 28a is a perspective view of an additional alternative embodiment one-piece container including triangular compartments forming a square, and a warmer;

FIG. 28b is a perspective view of a further alternative embodiment one-piece container forming a circle, and a warmer;

FIG. 28c is a perspective view of another alternative embodiment one-piece container forming a triangle, and a warmer;

FIG. 29a is a side view of an additional embodiment container and warmer having exterior lights;

FIG. 29b is a perspective view of another alternative embodiment container having interior lights;

FIG. 30 is an exploded perspective view of a container having a lid;

FIG. 31a is an exploded view of an alternative embodiment one-piece container and warmer, with a lid;

FIG. 31b is a side cross-sectional view of the container and warmer of FIG. 31a;

FIG. 32a is a side view of an alternative embodiment container and warmer;

FIG. 32b is a side view of an additional alternative embodiment container and warmer;

FIG. 32c is a side view of a further alternative embodiment container and warmer;

FIG. 32d is a side view of another alternative embodiment container and warmer;

FIG. 32e is a perspective view of an additional alternative embodiment container and warmer;

FIG. 33a is a side view of an alternative embodiment stacked container set;

FIG. 33b is a perspective view of a warmer of FIG. 33a;

FIG. 34 is a perspective view of an alternative embodiment container having a decorative divider;

FIG. 35a is perspective view of a retainer for use with a container;

FIG. 35b is a perspective view of a container and an alternative embodiment retainer with decorations;

FIG. 35c is a perspective view of an additional alternative embodiment decorative retainer;

FIG. 36 is a perspective view of an alternative embodiment multi compartment, multi piece container having a mechanism to connect the container portions;

FIG. 37 is a perspective view of a multi compartment, multi piece container and a retainer clip;

FIG. 38 is a perspective view of a multi compartment, multi piece container and an alternative embodiment decorative retainer clip;

FIG. 39 is a perspective view of a multi compartment, multi piece container and an alternative embodiment decorative retainer and retainer clip;

FIG. 40 is an exploded perspective view of a multi compartment, multi piece container and an alternative embodiment magnetic retainer clip;

FIG. 41 is a perspective view of a multi compartment, multi piece container and a plurality of magnetic retainer clips;

FIG. 42 is a perspective view of a multi compartment, multi piece container and alternative embodiment retainer clips;

FIG. 43a is a perspective view of a multi compartment, multi piece container and a further alternative embodiment retainer clip;

FIG. 43b is a perspective view of an alternative embodiment retainer clip;

FIG. 44a is an exploded perspective view of warmer having a heat conductor and a compartment of a multi compartment, multi piece container;

FIG. 44b is a top view of the warmer and heat conductor of FIG. 44a;

FIG. 45a is a perspective view of a multi compartment, multi piece container and an alternative embodiment conductor having a receptacle;

FIG. 45b is a perspective view of the conductor of FIG. 45a;

FIG. 46 is a perspective view of an alternative embodiment heat conductor having an illumination mechanism;

FIG. 47 is a perspective view of an additional embodiment heat conductor;

FIG. 48a is a perspective view of a further embodiment heat conductor and a corresponding compartment of a multi compartment, multi piece container;

FIG. 48b is a perspective view of an additional embodiment heat conductor and a corresponding one piece container;

FIG. 49 is a perspective view of an alternative embodiment cover with multiple containers and warmers;

FIG. 50 is a perspective view of an alternative embodiment compartment of a multi compartment container;

FIG. 51a is a bottom view of an alternative embodiment cover having a mechanism for allowing manual winding of an electrical cord;

FIG. 51b is a bottom view of an additional alternative embodiment cover having a mechanism for allowing automatic winding of an electrical cord;

FIG. 52a is a side view of an alternative embodiment cover on a warmer, the cover having openings to allow the passage of light and/or aroma; and

FIG. 52b is a side view of an additional alternative embodiment cover on a warmer, the cover having a light source and openings to allow the passage of light and/or aroma.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

It must be noted that, as used in this specification and the appended claims, the singular forms “a,” “an,” and “the” include plural referents unless the context clearly dictates otherwise. Also, as used herein, the terms “comprising,” “including,” “containing,” “characterized by,” and grammatical equivalents thereof are inclusive or open-ended terms that do not exclude additional, unrecited elements or method steps.

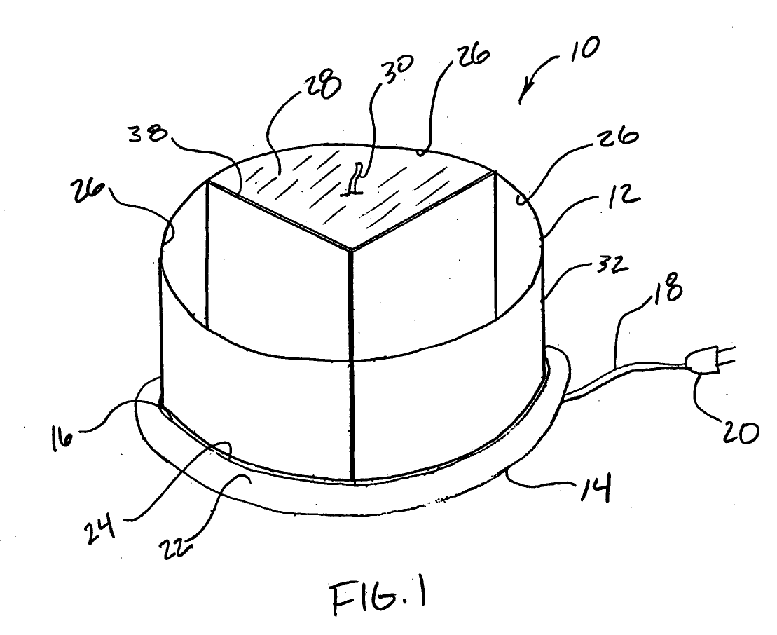

Referring now to FIG. 1 a perspective view is shown of an apparatus for producing an aroma, indicated generally at 10. The apparatus 10 may include a container 12 and a warmer 14. The warmer 14 may include any of a variety of devices known in the art for producing heat. For example, the warmer 14 may include a heating surface or plate portion 16 that may be configured to generate heat using electrical power. One embodiment of the warmer 14 may be formed as a hot plate that generates heat by electrical resistance. The warmer may have an electrical cord 18 and a connector 20 to be received in an electrical outlet (not shown) in a manner known in the art. It will be understood that any other heat generating mechanism known in the art, such as a heat lamp or blower for example, may be used in connection with the warmer 14 within the scope of the present disclosure.

One embodiment of the warmer 14 may include a ridge 22 at least partially circumscribing the plate 16 to define a recess 24 for receiving the container 12 to assist in positioning and maintaining the container 12 on the warmer 14. The ridge 22 may be formed as a wall, bump, or any variety of upward projecting members. It will be understood that other embodiments of the warmer may be formed without the ridge 22 such that the warmer 14 may define a planar surface. Moreover, the warmer 14 may include discontinuous and/or non-planar heating surfaces as an alternative to the plate 16.

The embodiment of the container 12 in FIG. 1, may include a plurality of portions or compartments 26. The compartments 26 may be configured for receiving a fragrance producing medium 28. The fragrance producing medium 28 may include various materials known in the art for producing a fragrance, such as scented candles, including votive candles, pillar candles and jar candles, for example. Also, the fragrance producing medium 28 may include materials such as scented candle wax, candle tarts, wax chips, wax potpourri, potpourri, scent chips, aromatic oils, essential oils, aromatic gels, or any other solid fragrance or liquid fragrance material known in the art. Accordingly, the fragrance producing medium 28 may include a variety of substances that generate a pleasant fragrance, and the amount of fragrance produced may be increased as the medium 28 is heated. It will be understood that the fragrance producing medium 28 may be formed of a substance that may not easily spoil and is not typically eaten by humans, though the container 12 may be used in other situations to contain edible substances.

One embodiment of the present disclosure may include compartments 26 forming a candle having a wick 30 such that the wick 30 may be burned in a manner known to those skilled in the art. Alternatively, the compartment 26 may be placed on the warmer 14 such that the heat generated by the warmer 14 may be used to increase the fragrance emitted from the fragrance producing medium 28 without the need of lighting the wick 30. This may provide an advantage of increased safety in that a flame is not required to produce a fragrance such that the danger of unwanted fire or injury may be reduced. Also, heating of the fragrance producing medium 28 without a flame may reduce unwanted emissions of contaminants into the air. This may reduce irritation of the eyes and lungs of users, for example, and may allow a fragrance to be generated in a clean manner.

One advantage of the container 12 of the present disclosure, is that a custom aroma may be developed by placing a different fragrance producing medium 28 in the compartments 26 of the container 12. For example, a first fragrance producing medium 28 may be placed in a first compartment 26 and a second, different scented fragrance producing medium 28 may be placed in a second compartment 26 such that a blended aroma may be produced without actually mixing the fragrance producing media 28. In one embodiment of the present disclosure, the compartments 26 may be moveable with respect to each other such that various different combinations of compartments 26 may be created and placed on a warmer 14 to generate a desired aroma. For example, in the embodiment of the container of FIG. 1, if each of the three different compartments 26 contains a different fragrance producing medium 28, the three compartments 26 may be placed on the warmer 14 in seven different arrangements to produce four blended aromas in addition to the three individual fragrances. Additional compartments 26 containing additional fragrance producing media 28 may also be combined to create even further blends of aromas, thereby increasing the possible aromas produced and enhancing the versatility of the container 12. Accordingly, various custom blended aromas can be created to suit an individual user.

The separate compartments 26 provide additional advantages in that the ease and safety of producing a blended aroma may be enhanced since the fragrance producing media 28 need not be actually mixed. It will be understood that a single fragrance producing medium 28 may be placed in a compartment 26, or alternatively if desired, a plurality of compatible different scented fragrance producing media 28 may be placed in a single compartment 26 to produce a blended aroma in the compartment 26. Moreover, the enhanced flexibility of the container 12 with separate compartments 26 may allow a reduction in the waste of fragrance producing media 28. For example, if the fragrance producing medium 28 in any compartment 26 becomes undesirable, the other compartments 26 may continue to be used.

It will also be understood that the compartments 26 may also be combined in different configurations without placing the compartments 26 on the warmer 14. For example, one embodiment of the present disclosure may include an evaporative gel as the fragrance producing medium 28, such that the warmer 14 may not be needed to produce an aroma. Accordingly, the compartments 26 may contain the fragrance producing medium 28 for producing a blended or unitary aroma by burning a wick 30 or for use without the enhanced aroma producing effects of the warmer 14.

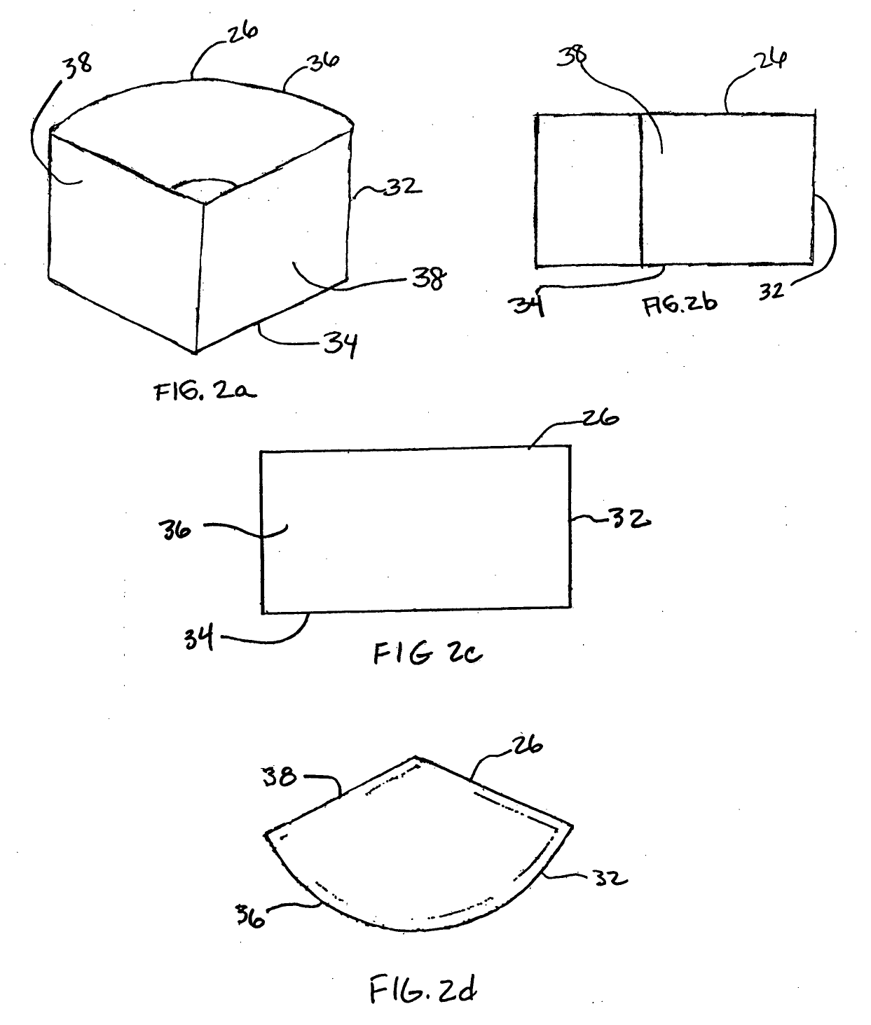

As shown most clearly in FIGS. 1-2d, the compartments 26 may be formed with a sidewall 32 and a bottom portion 34. In one embodiment, the sidewall 32 may extend in a substantially perpendicular orientation with respect to the bottom portion 34. The bottom portion 34 may define a substantially planar portion for contacting the warmer 14. The sidewall 32 may include a divider 38 that may be configured to mate with another compartment 26 to form an interior portion of the container 12. As used herein, the term “mate” shall be construed broadly to include dividers 38 that are configured to join or pair, whether in fixed or removable contact or in non-contacting close proximity, such that a divider portion 38 of a compartment 26 may correspond with another divider portion 38 of a different compartment 26. The divider portions 38 may or may not be attachable to each other, and may have similar or different sizes and shapes. Accordingly, a divider portion 38 may be configured to contact a corresponding divider portion 38, or at least a portion of a divider portion 38 may be spaced apart from a corresponding divider portion 38. The sidewall 32 may also include an exterior portion 36 that may not necessarily be configured to join with another compartment 26, but may form an exterior of the container 12.

One embodiment of the container 12 may include a plurality of compartments 26 that may be substantially identical in size and shape. The compartments 26 may have divider portions 38 that may be configured to contact adjacent divider portions 38 over substantially an entire surface area of the divider portions 38. This may allow the compartments 26 to be joined together to form the container 12 such that the container 12 may have the appearance of a unitary member. Other embodiments of the present disclosure may include compartments of different sizes and shapes having divider portions that do not completely contact adjacent divider portions.

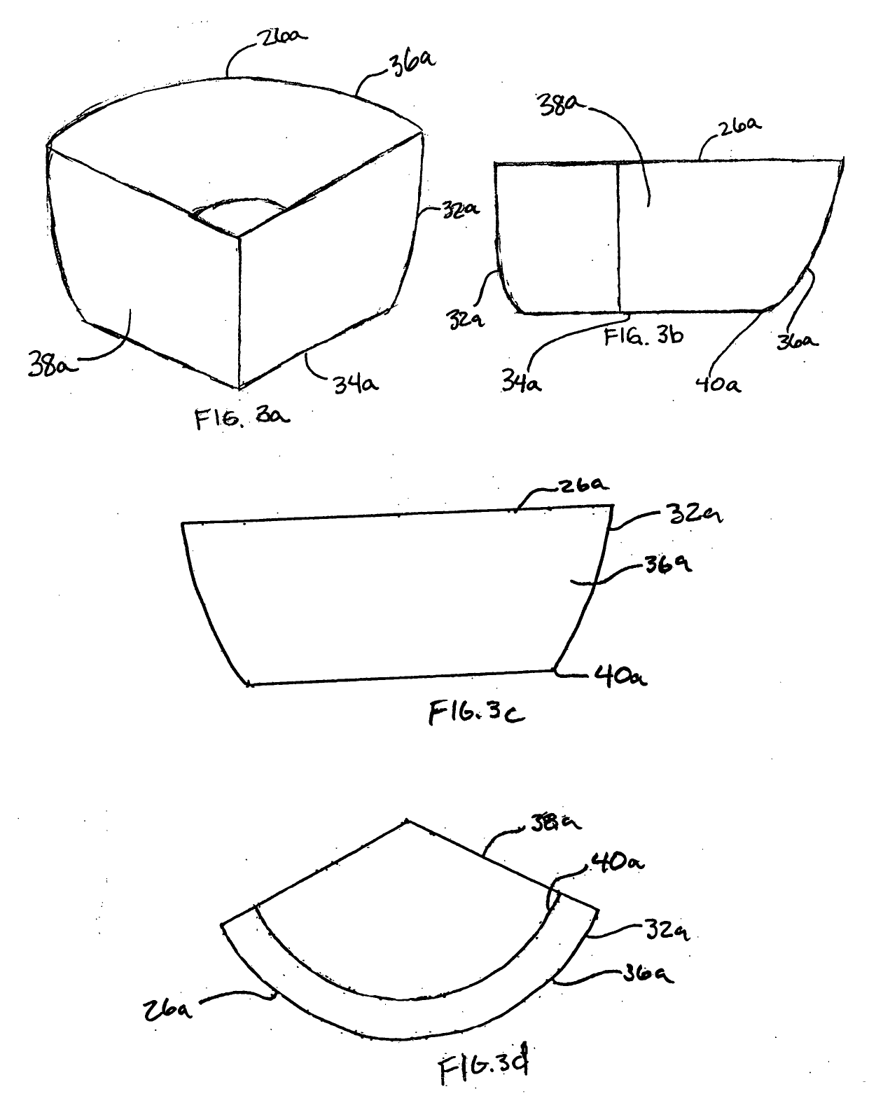

As shown in FIGS. 3a-3d, an alternative embodiment of a compartment 26a may be formed with a convex exterior portion 36a such that at least a portion of the sidewall 32a extends laterally beyond a perimeter 40a of the bottom portion 34a. As used herein, the term “laterally” refers to situations in which the sidewall 32a extends in a direction such that at least a vector component of the direction extends in a lateral direction, i.e. sideways or slanted up. This may allow the compartment 26a to have an increased volume and a variety of shapes while still remaining compatible with a particular warmer 14. Also, by extending at least a portion of the sidewall 32a beyond the perimeter 40a of the bottom portion 34a, the warmer 14 may be less visible and the contents of the compartment 36a may be easier to remove.

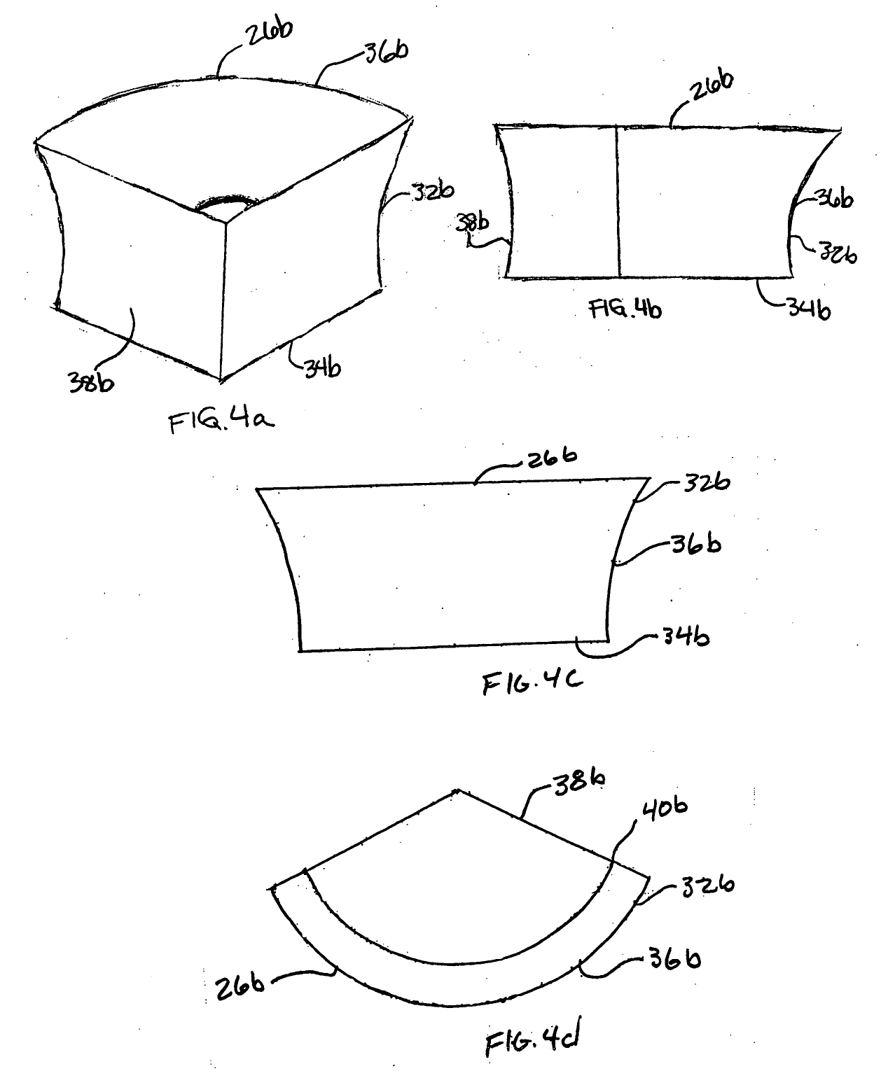

As shown in FIGS. 4a-4d, an additional embodiment compartment 26b may be formed with a concave exterior portion 36b. It will be understood that the sidewall 32 may be formed in various other configurations, such as an angled configuration, or any combination of straight, angled and/or curved portions, or any other decorative or ornamental shape, for example, within the scope of the present disclosure.

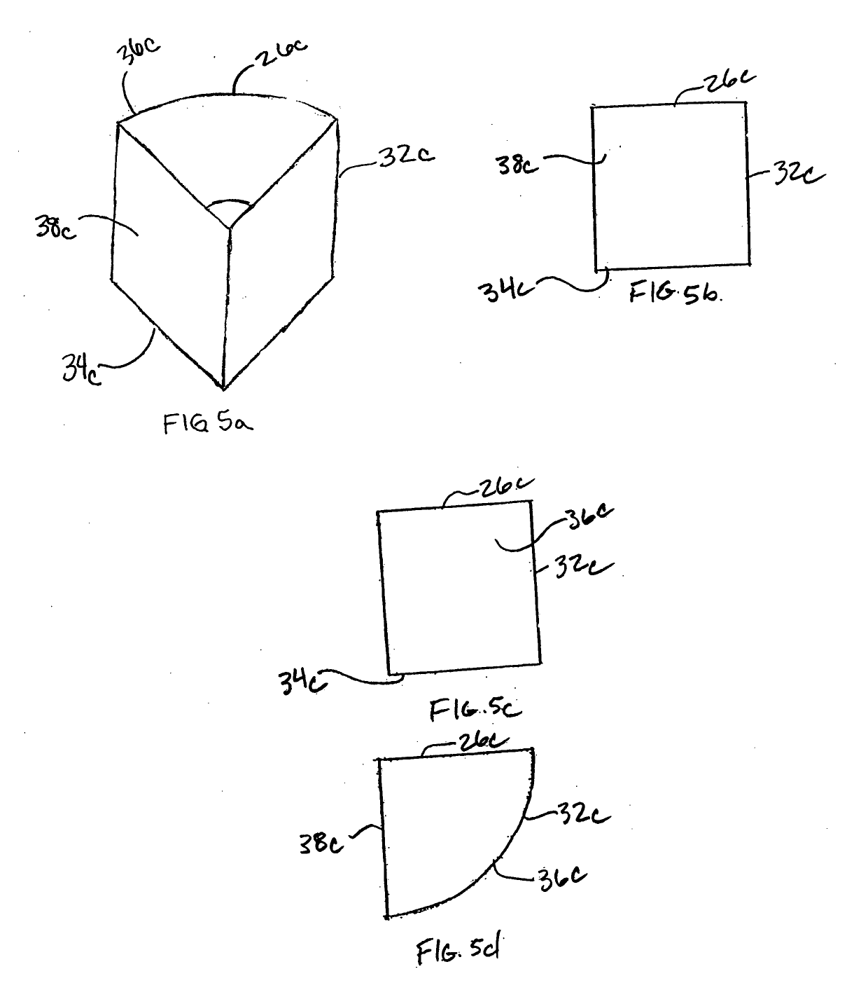

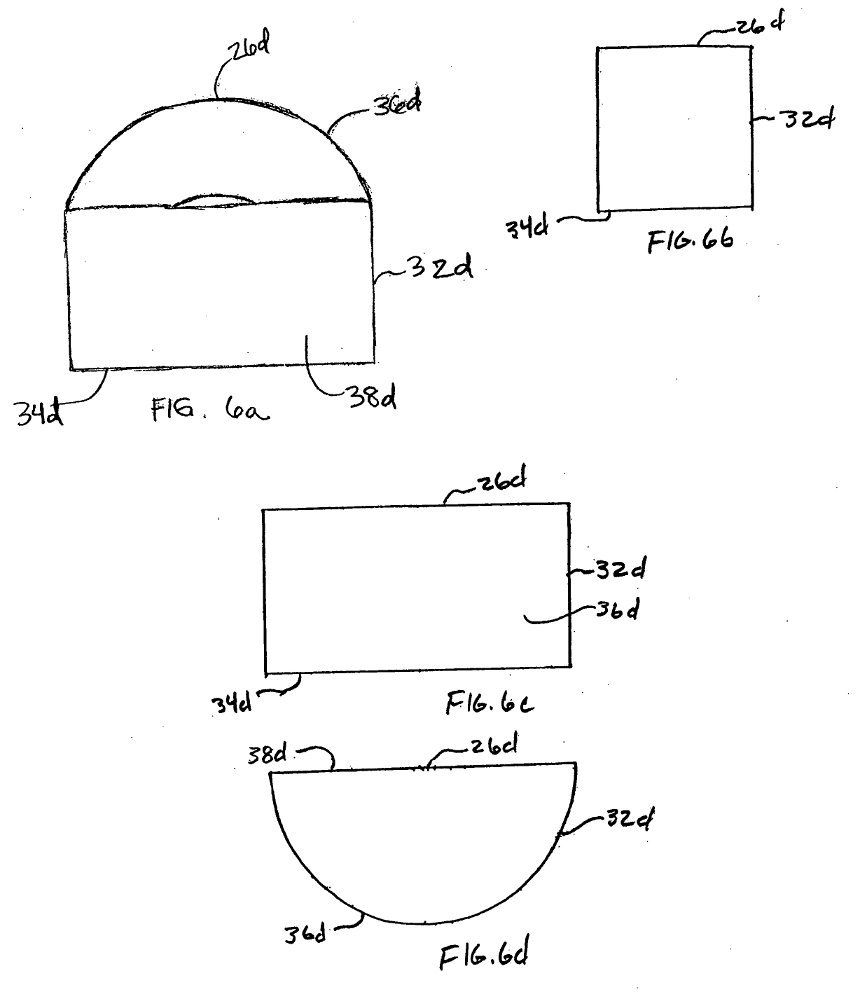

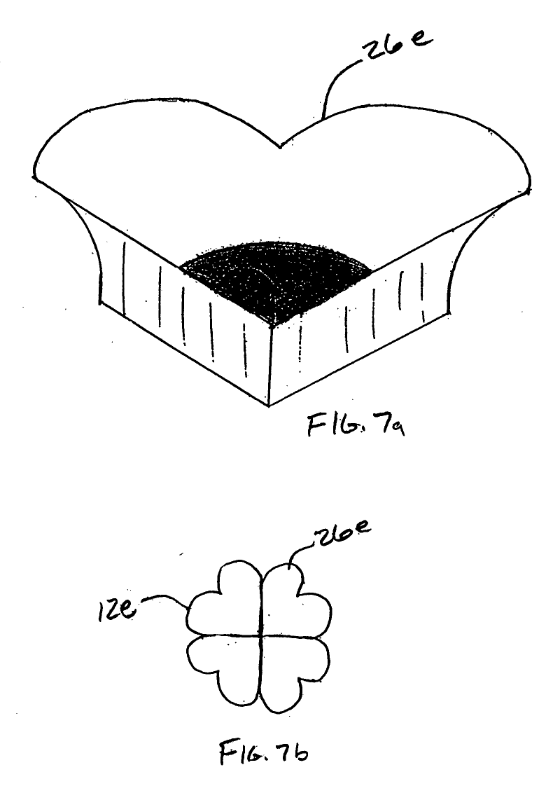

Referring now to FIGS. 5a-5d, an alternative embodiment compartment 26c is shown. It will be understood that the alternative embodiment compartment 26c may be configured such that four compartments 26c may be combined to form a container, rather than the three compartments 26 in the container 12. Moreover, it will be understood that the principles of the present disclosure may be utilized with any number of compartments to form a container, including a half container compartment 26d, as shown in FIGS. 6a-6d.

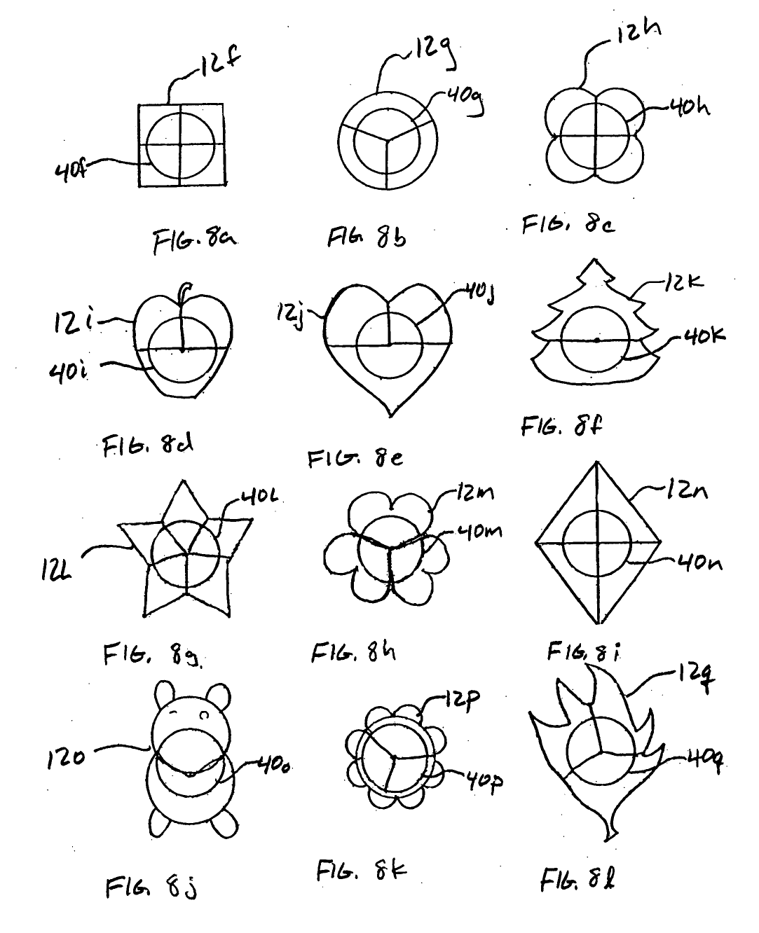

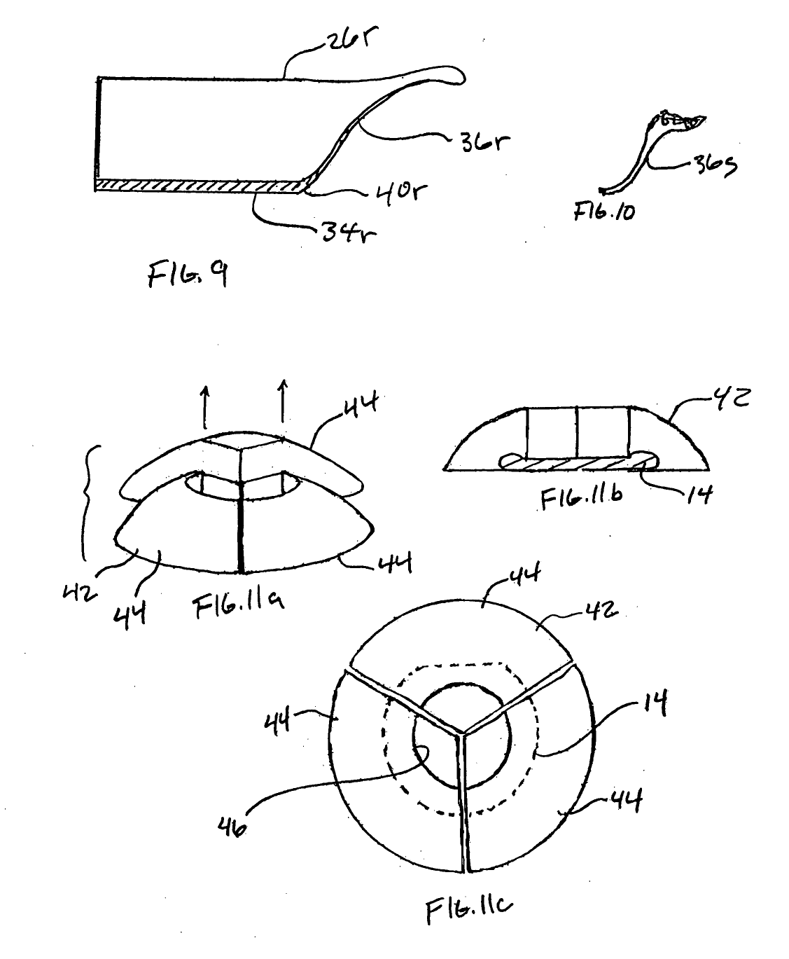

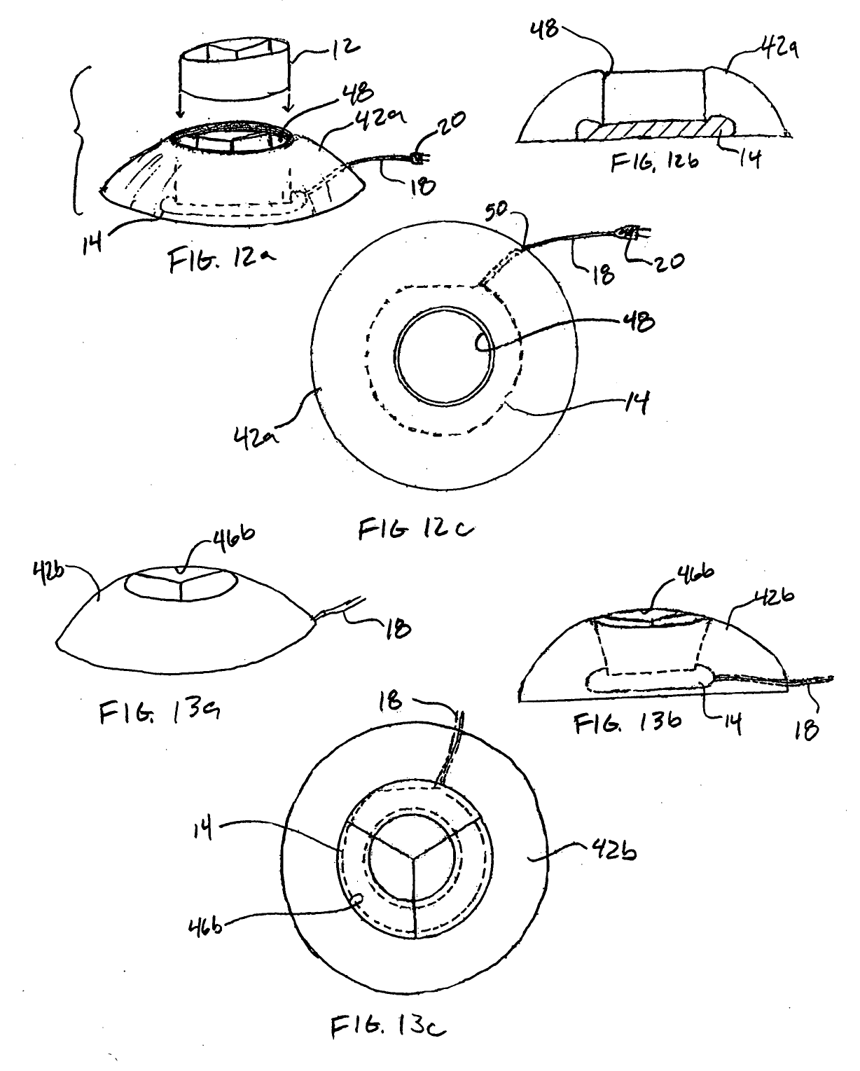

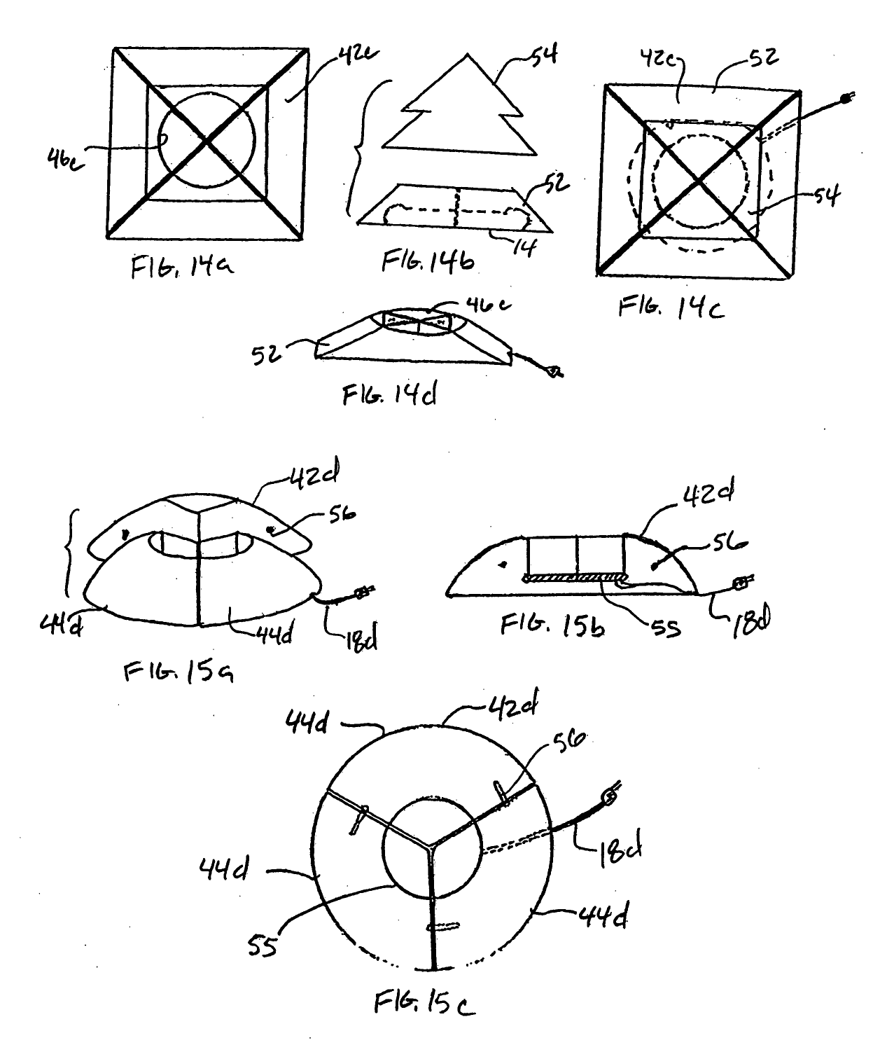

Accordingly, it will be understood that any number of compartments having various different configured sidewalls and shapes may be used to form a container within the scope of the present disclosure. Moreover, the container itself may be formed in various different shapes. For example, as shown in FIGS. 7a and 7b, a container 12e may be formed of a plurality of heart-shaped compartments 26e, or as shown in FIGS. 8a-8l, containers 12f-12q, may be formed in various other shapes, such as square, round, clover, fruit, heart, tree, star, triple heart, diamond, animal, flower, or leaf shapes, respectively, for example. Moreover, the container may be formed in any other shape within the scope of the present disclosure. It will be understood that the embodiments of the container in FIGS. 8a-8l may include a bottom perimeter 40f-40q, respectively, that may be configured to correspond to a recess 24 on a warmer 14. The perimeter 40f-40g may be round or any other shape in accordance with the principles of the present disclosure.

Referring to FIG. 9, a side cross-sectional view is shown of an additional embodiment compartment 26r having a bottom portion 34r that may be thicker and/or weighted to be heavier than the sidewall 32r, to assist in stabilizing and holding the compartment 26r within the recess 24 of the warmer 14 or on any other surface. Thus, the weight of the bottom portion 34r may compensate for the weight of the exterior portion 36r. Accordingly, the exterior portion 36r that extends beyond the perimeter 40r may be supported by the bottom portion 34r without causing the compartment 26r to tip. The compartment 26r may therefore be configured to remain stable even though the exterior portion 36r may form an overhang.

It will be understood that the exterior portion 36r may be formed without any decorative ornamentation, or as shown in FIG. 10, an exterior portion 36s may be formed with decorative ornamentation. It will be appreciated that any variety of ornamentation may be used within the scope of the present disclosure. Moreover, any portion of the compartment 26 may include ornamentation within the scope of the present disclosure. Also, it will be understood that the structure depicted in FIG. 10 may form a handle and that handles may be positioned on the containers of the present disclosure in various different arrangements.

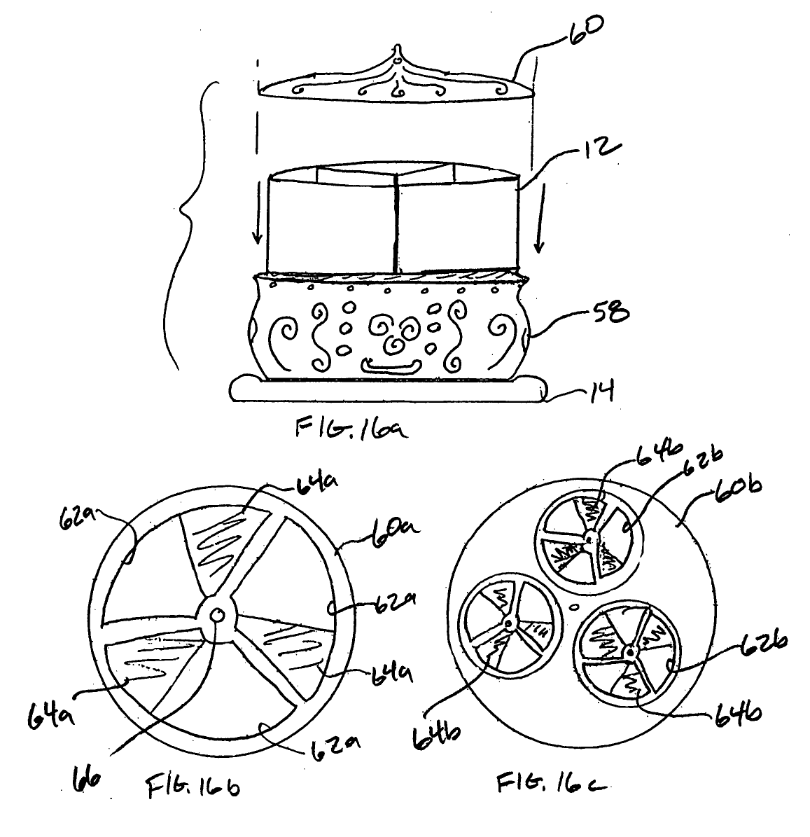

Referring now to FIGS. 11a-11c, a cover 42 is shown for use in covering a warmer 14 to conceal the warmer 14 and allow decor variations to be applied to the warmer 14. Accordingly, a single warmer 14 may be used with various different decorative themes for multiple occasions. The cover 42 may be formed in sections 44, such that the cover 42 may be formed of a plurality of parts. For example, the cover 42 may be formed with three sections 44 as shown in FIGS. 11a-11c, or the cover 42 may be formed with any other quantity of sections. It will be appreciated that the cover 42 may be formed in any shape including dome or box shapes, for example, and may define a space for receiving the warmer 14. The cover 42 may be formed as a substantially hollow member such that a majority of the volume within the cover 42 is available for receiving the warmer 14, or the cover 42 may be formed as a substantially solid member with a space for the warmer 14 defined in the cover 42 such that a majority of the space in the cover 42 is occupied by the warmer 14. Moreover, each section 44 may include a chamber 46 for use in receiving a fragrance producing medium 28 or a compartment 26 of a container 12. It will also be understood that the cover 42 may be formed with a built-in warmer such that the cover 42 and warmer 14 may be configured as a unitary appliance.

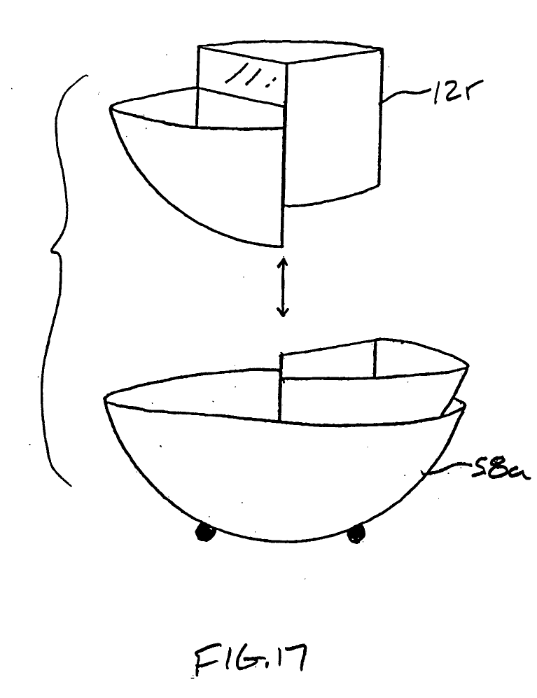

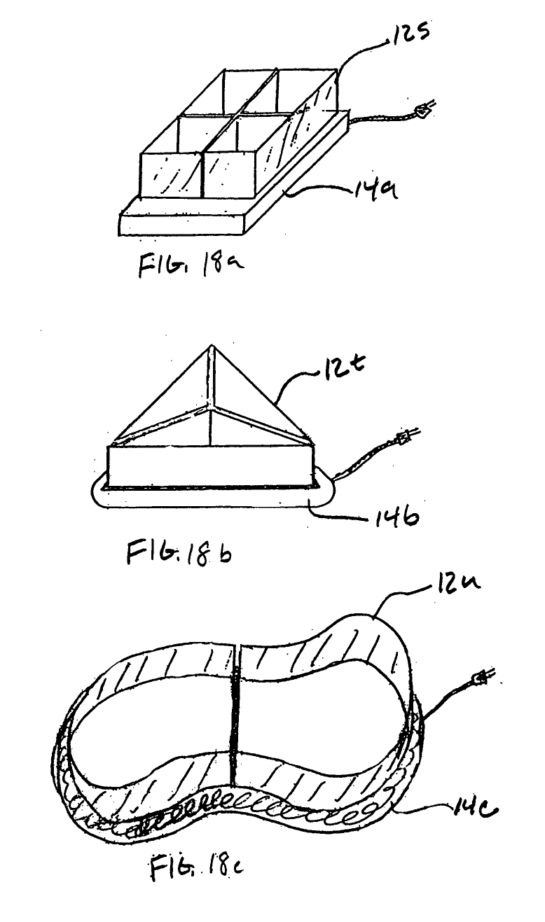

As shown in FIGS. 12a-12c, a cover 42a may be formed as a one piece unit having an opening 48 formed therein. Accordingly, the cover 42a may be formed without chambers 46 such that a container 12 may be received in the opening 48. The opening 48 may be formed in any shape and size to be compatible with a desired container 12 and warmer 14. A groove 50 may be formed in the cover 42a for allowing the cord 18 of the warmer 14 to pass outside the cover 42a, and allowing the cover 42a to remain flush with a support surface, such as a counter top. Also, as shown in FIGS. 13a-13c, the cover 42b may be formed of a single piece, and the cover 42b may have multiple chambers 46b integrally formed therein.

It will be understood that various other shapes and configurations may be used to form a cover. For example, as shown in FIGS. 14a-14d, a cover 42c may be formed in the shape of an object, such as a tree, having a base 52 and a lid 54. The lid 54 may be placed on the base 52 such that the chamber 46 may be concealed. Accordingly, it will be understood that the portions forming the cover 42c may be stacked upwardly as well as laterally.

As shown in FIGS. 15a-15c, a cover 42d may be provided in which the sections 44d may be electrically coupled together to generate heat in a heater 55 in one or more of the sections 44d. For example, one of the sections 44d may include a cord 18d for conveying electrical power to a heater 55 in the section 44d. Electrical connectors 56, such as a projection and/or a socket may be provided on the sections 44d such that electrical power may be conveyed to the heaters 55 in the other sections 44d to generate heat in the other sections 44d. Thus, one or more of the heaters 55 in the sections 44d may be used to generate heat for use with a fragrance producing medium 28. It will be understood that any variety of electrical connectors 56 known in the art may be used to allow multiple sections 44d to be connected or detached such that a desired heating configuration of the cover 42d may be accomplished.

Referring now to FIGS. 16a-16c, an additional embodiment of the present disclosure is shown. The embodiment of the disclosure depicted in FIG. 16a may include a container or bowl 58 in any variety of configuration and having any variety of ornamentation. It will be understood that in the present embodiment, as well as the other embodiments of the present disclosure, the bowl 58 may be mottled, etched, frosted, glazed, textured, painted or include decorative holes, for example, to provide decorative ornamentation in the bowl 58. The bowl 58 may be placed on a warmer 14 or the bowl 58 itself may include a warming mechanism such that the warmer 14 is not necessary. A container 12 may be received in the bowl 58 and a bowl lid 60 may be placed over the bowl 58 to conceal the container 12. It will be understood that the lid 60 may also include any variety of ornamentation and may be provided as a matching set with the bowl 58.

As shown in FIG. 16b, a lid 60a may include one or more openings 62a to allow fragrance to be emitted from the bowl 58. The lid 60a may also include one or more stops 64a for closing the openings 62a. In one embodiment, the stops 64a may operate in unison such that the openings 62a may all be opened or closed at the same time. For example, the stops 64a may be pivoted about an axis 66 so as to move to a position in which the openings 62a are open, or to a position in which the openings 62a are closed. Also, the openings 62a may be partially closed to control the amount of fragrance that may be emitted.

As shown in FIG. 16c, the bowl lid 60b may have stops 64b that may be independently adjustable so that any of the openings 62b may be opened or closed independently with respect to the other openings 62b. It will be understood that openings and stops of various different quantities, configurations and arrangements may be used in accordance with the principles of the present disclosure.

Referring now to FIG. 17, another alternative embodiment of the present disclosure is shown in which a bowl 58a is provided for receiving a semi-spherical container 12r. As discussed above, the bowl 58a may be configured for placing on a warmer 14, or the bowl 58a may be configured to have a warming apparatus incorporated integrally into the bowl 58a. It will be understood that the warmer 14 may be formed in any shape and configuration within the scope of the present disclosure. For example, as shown in FIGS. 18a-18c, the warmer 14a-14c may be square, triangular, or curved shaped, respectively, or any other shape known to those skilled in the art. Similarly, the containers 12s-12u, may have corresponding square, triangular, or curved shapes compatible with the warmer. However, it will be understood that the containers may have different shapes, and in some embodiments, the shapes of the containers may not correspond to the shape of the warmer.

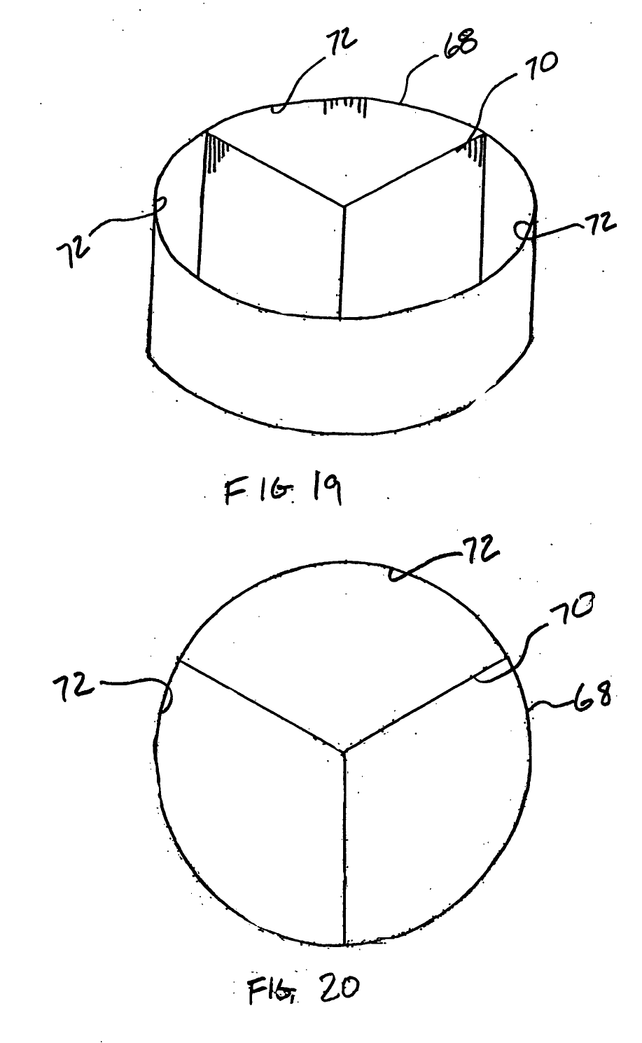

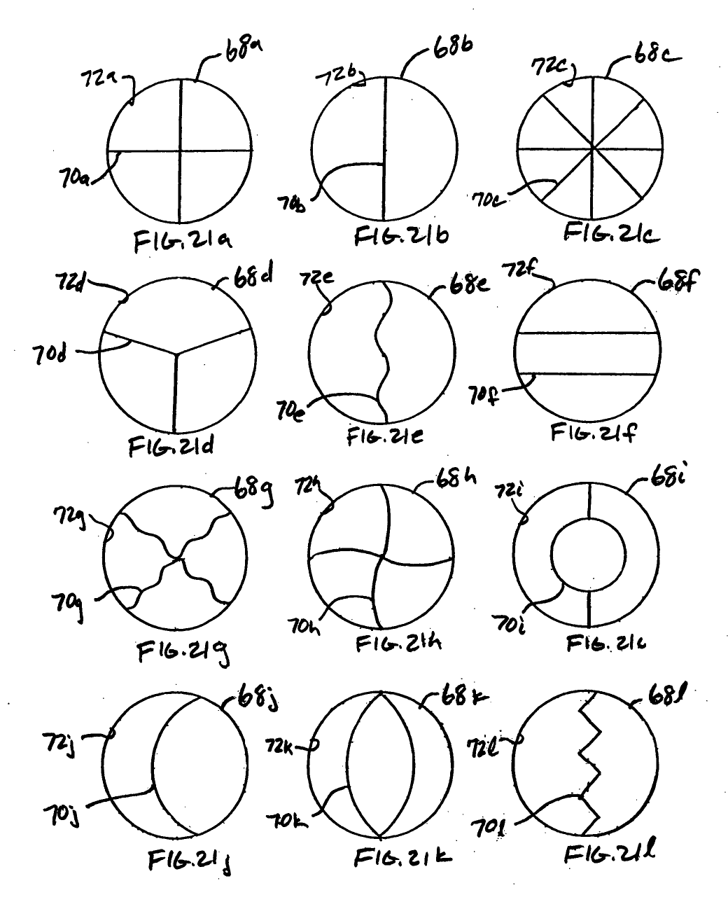

As shown in FIGS. 19 and 20, a one part container 68 may be provided within the scope of the present disclosure. The container 68 may include a fixed divider 70 that divides the container 68 into a plurality of fixed compartments 72. It will be understood that the one part container 68 may be used in a similar fashion as the multi-part container 12 discussed above, except that the compartments 72 may not be movable with respect to each other. Similar to the multi-part container 12, the one part container 68 may be specifically sized to mate with a warmer. This may be an improvement over prior art containers that are not specifically configured to mate with warmers such that the containers are less stable on the warmer and less efficiently warmed. It will be understood that the divider 70 may be arranged in any of a variety of configurations as depicted by dividers 70a-701, in FIGS. 21a-211. Moreover, other embodiments of the one part container 68 may have various different shapes besides the round shapes shown in FIGS. 21a-211.

As shown in FIG. 22, a one part container 68m may be placed in a bowl 58b, and the bowl 58b may be placed on a warmer 14 to warm a fragrance producing medium 28. Accordingly, in this embodiment of the disclosure, the one part container 68m may be shaped and sized to conform to the bowl 58b, and the bowl 58b may be shaped and sized to be compatible with the warmer 14. Alternatively, it will also be understood that a multi piece container may be placed in the bowl 58b within the scope of the present disclosure.

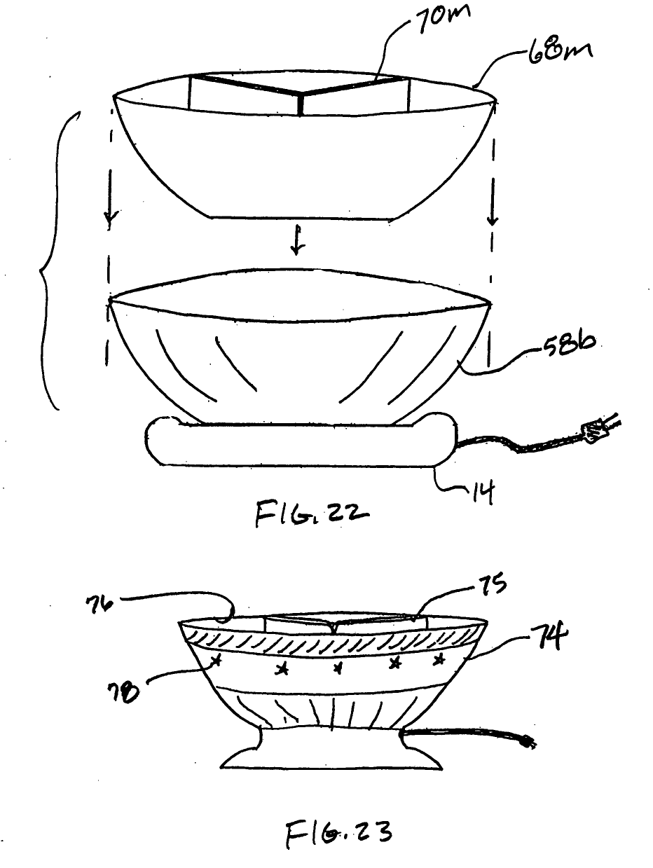

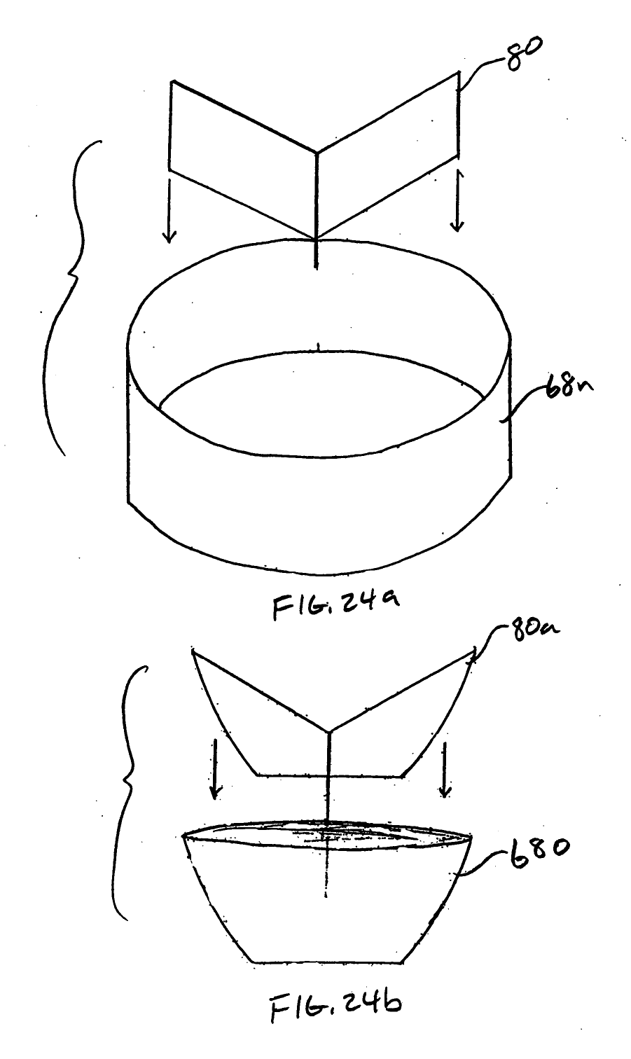

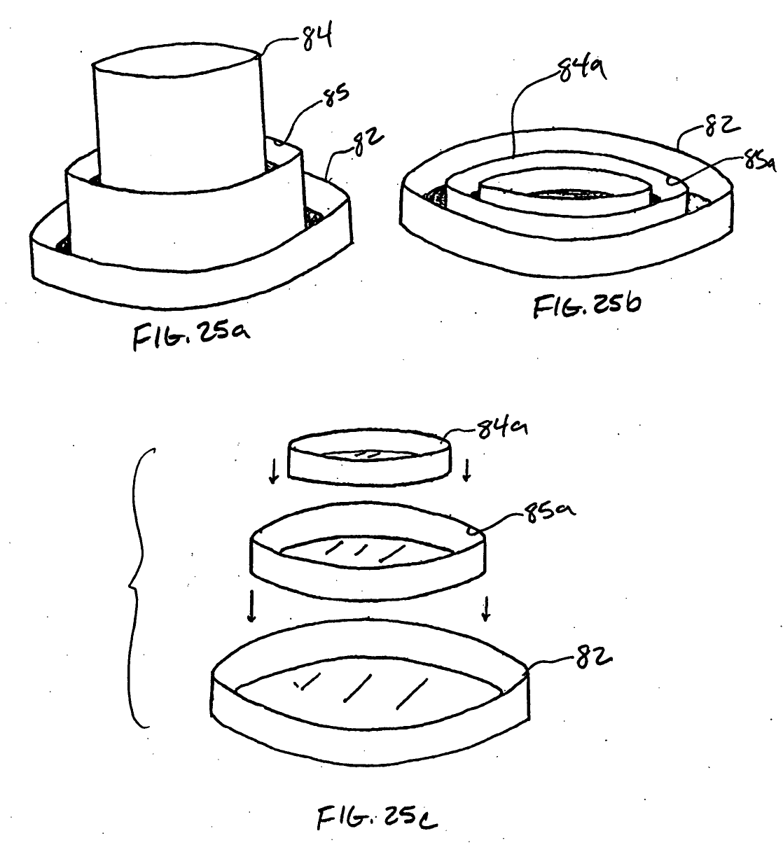

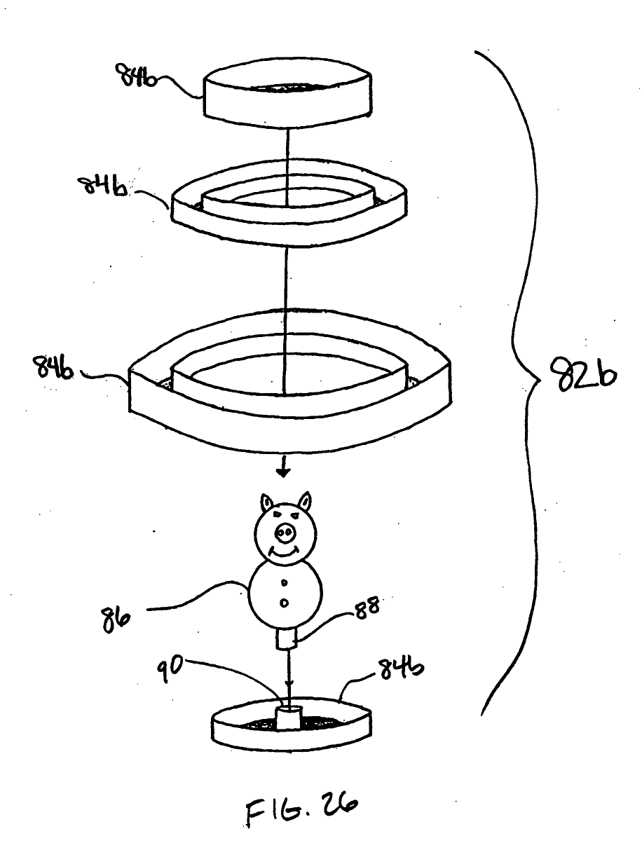

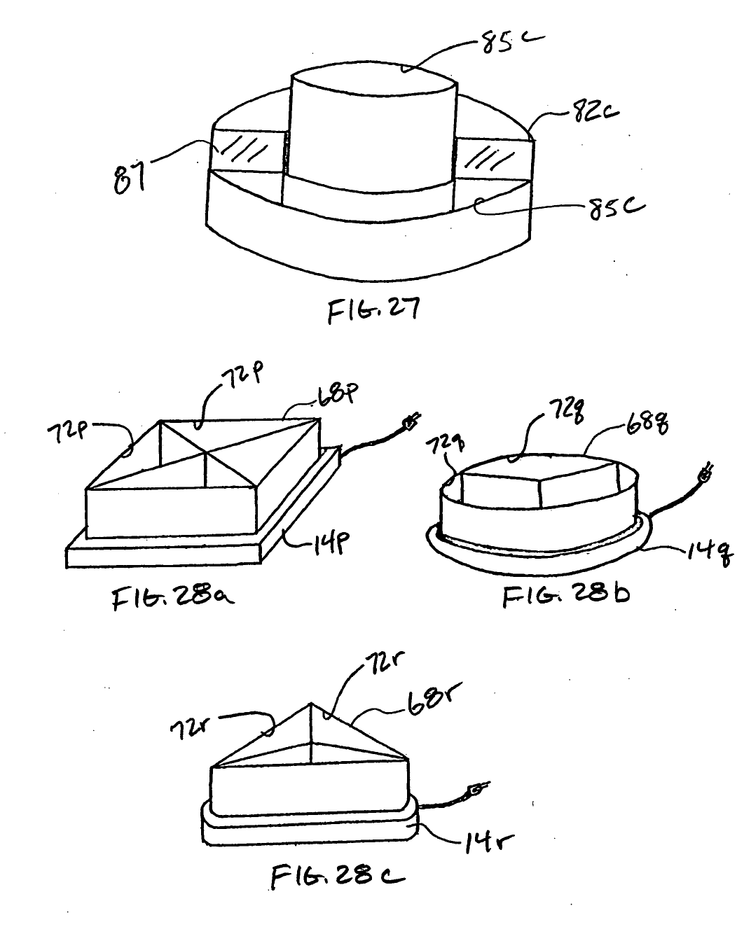

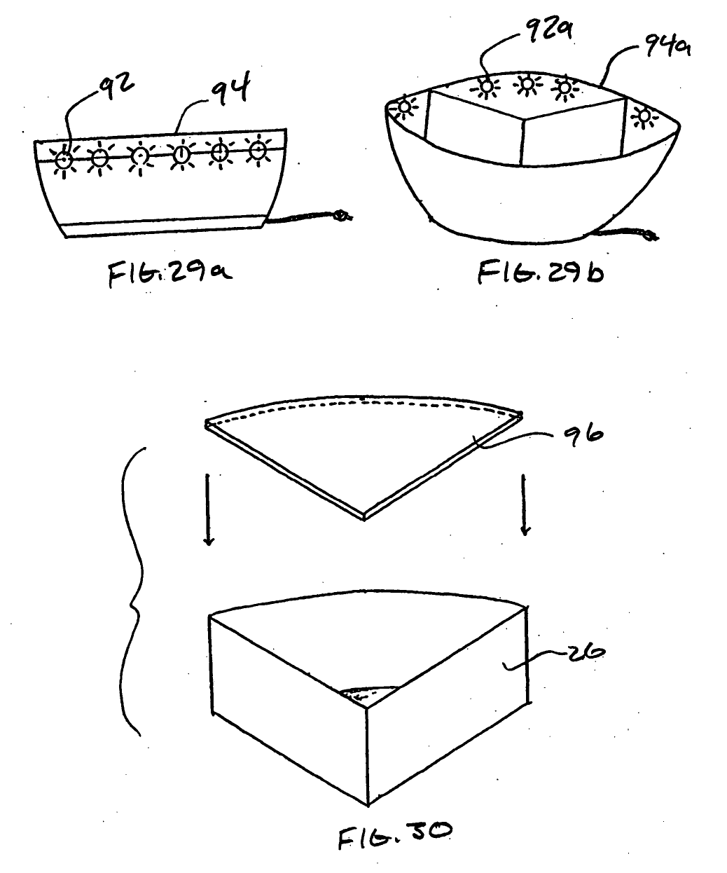

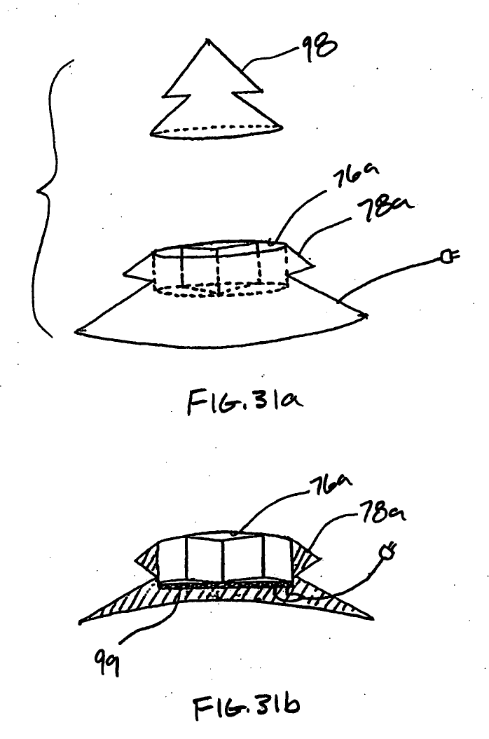

Alternatively, as shown in FIG. 23, a one part container, divider and warmer 74 may be provided in various different shapes, sizes and configurations. The one part container, divider and warmer 74 may be formed as a unit with a fixed divider 75 forming separated compartments 76. A warming mechanism may be formed integrally with the one part bowl 74 to form a unitary appliance. Similar to the previously discussed embodiments, the one part container 74 may have any of a variety of decorative ornamentation or indicia 78.

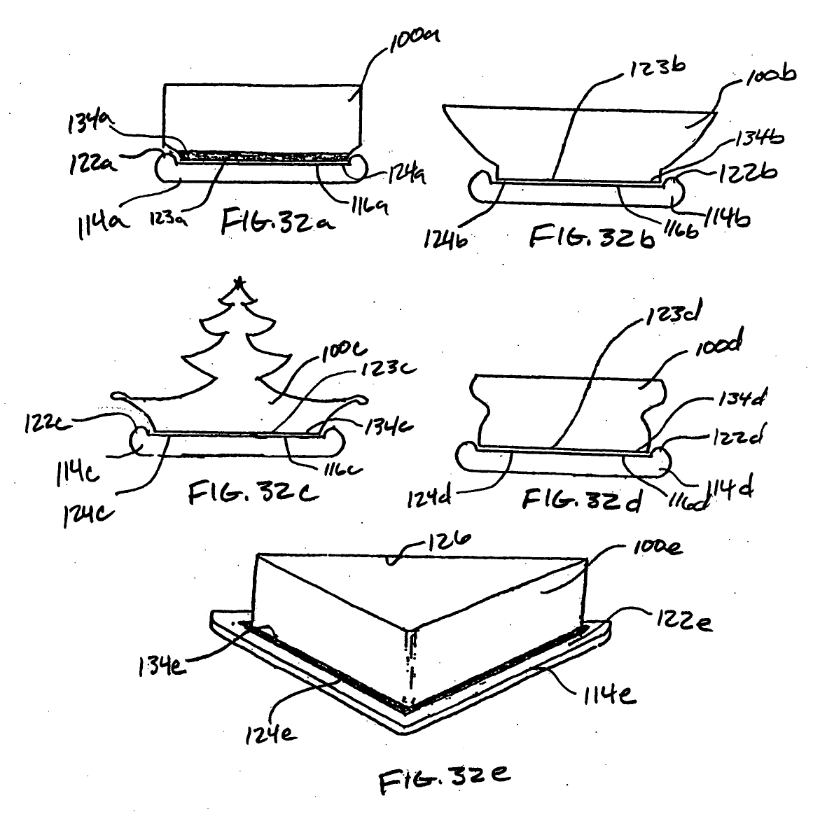

Referring now to FIG. 24a an additional embodiment is disclosed in which a container 68n having a single compartment may be divided in to a plurality of compartments with a modular divider 80. The modular divider 80 may be fixed in place with any of a variety of fastening means, such as an adhesive or fasteners, or the modular divider 80 may remain unattached to the container 68n. Moreover, the modular divider 80 may include any variety of sealing means to sealably attach the edges of the divider 80 to the container 68n to allow a fragrance producing medium 28 (shown in FIG. 1) to remain separated within the container 68n. It will be appreciated, as shown in FIG. 24b, that the container 680 may have various different shapes, sizes and configurations, and that the modular divider 80a may also be configured to correspond to the container 680. Similar to the other embodiments of the present disclosure, the modular dividers 80, 80a may be configured to provide any number of compartments in various different arrangements.

Referring to FIGS. 25a-25c, additional embodiments of the disclosure are presented in which a container 82 may be provided with concentric dividers 84 and 85, and 84a and 85a. The concentric dividers 84 may be provided in varying heights, or concentric dividers 84a may be provided with uniform heights. The concentric dividers 84, 84a, 85 and 85a may be fixed or modular and may be formed in various different shapes, such as circular, square, or any other shape. Accordingly, compartments may be formed by the dividers in rings in a concentric arrangement. Moreover, other embodiments of the present disclosure may be provided with similar shapes in which the dividers are positioned within the container 82 in a non-concentric arrangement.

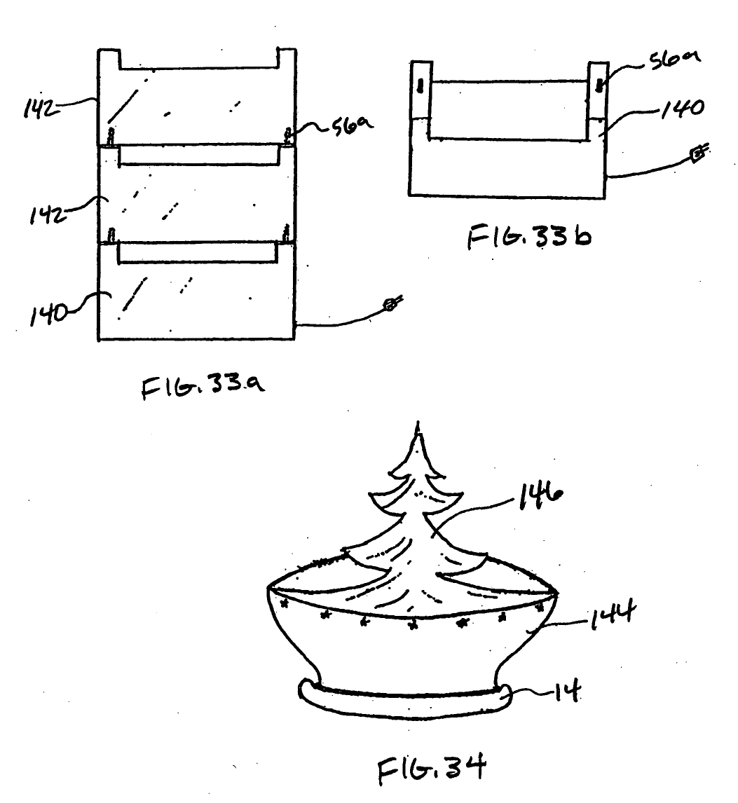

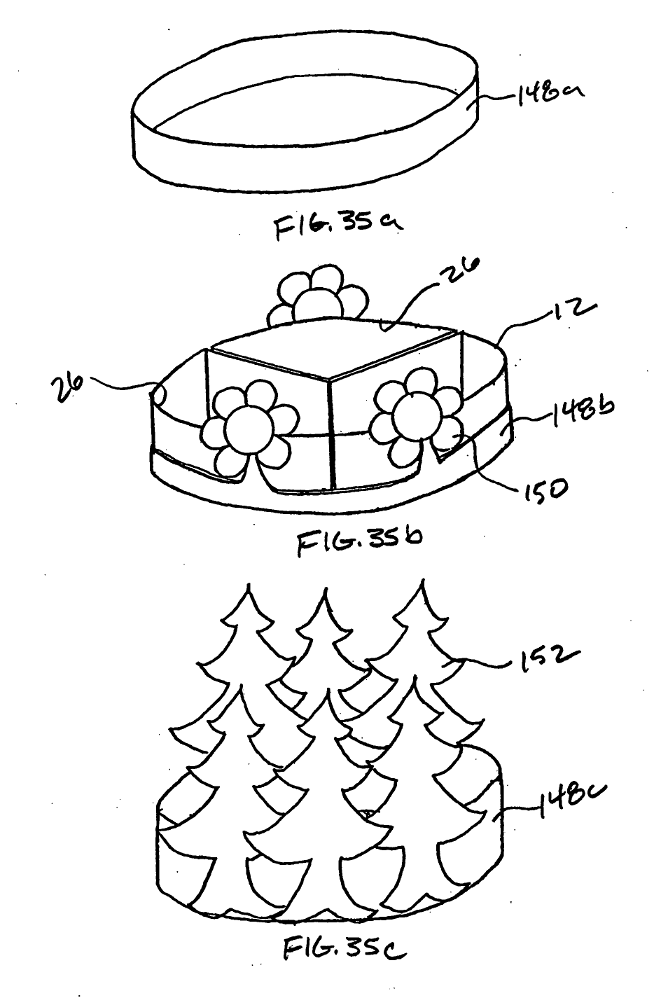

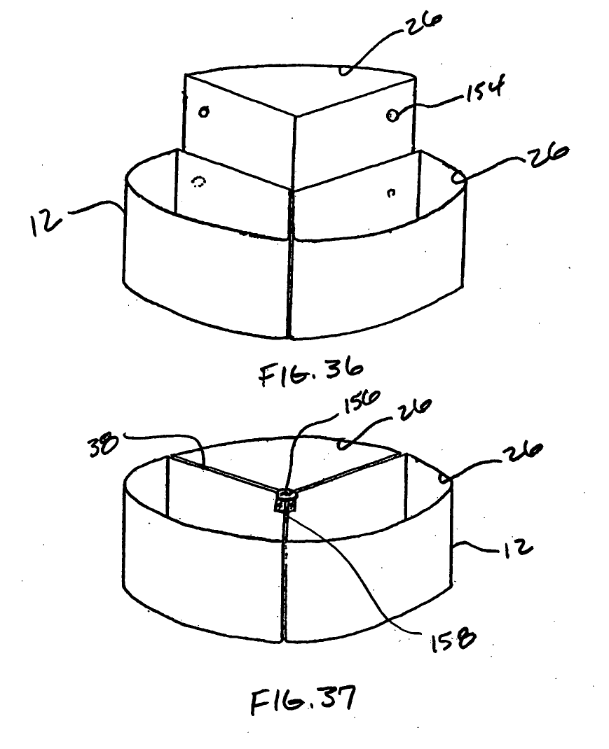

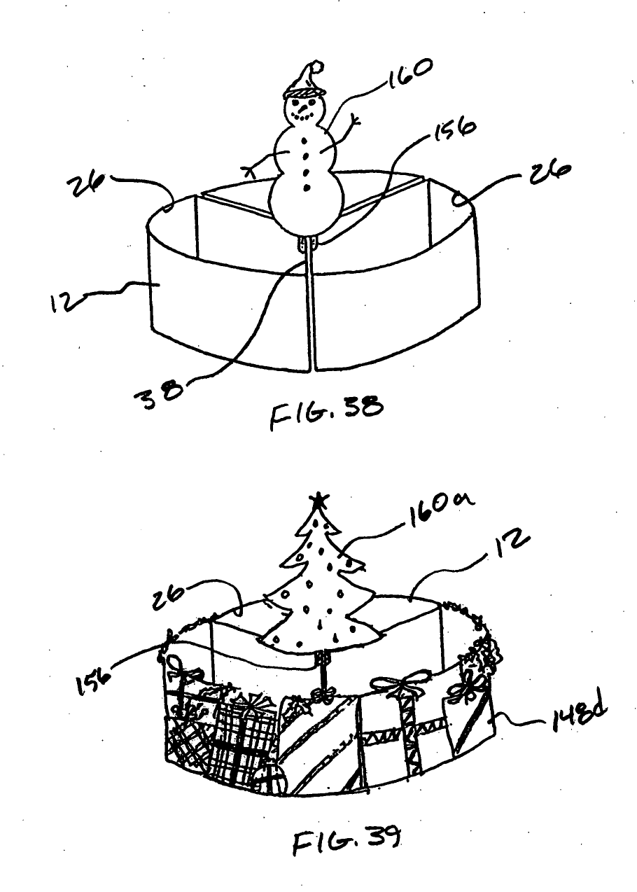

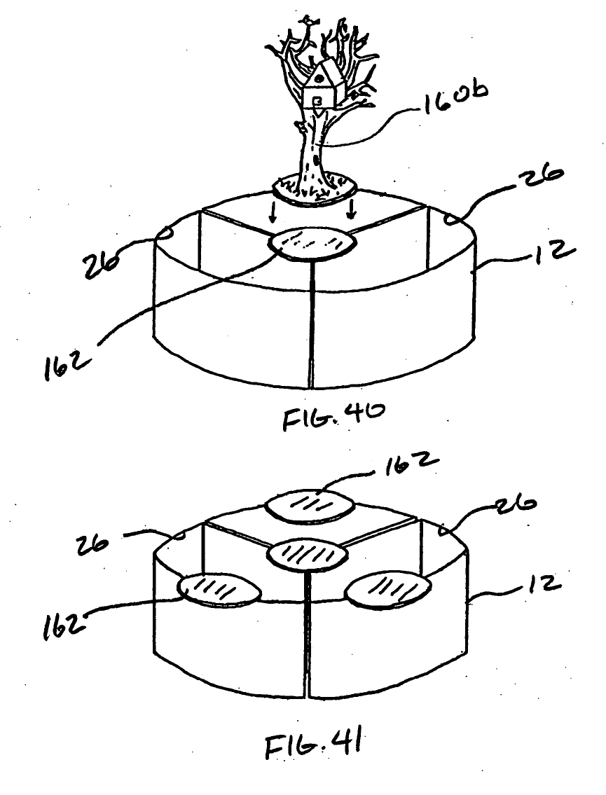

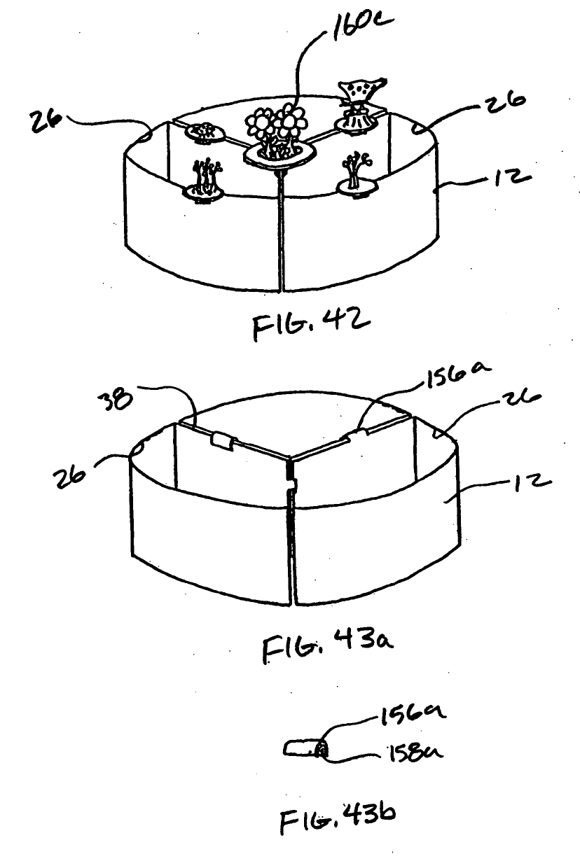

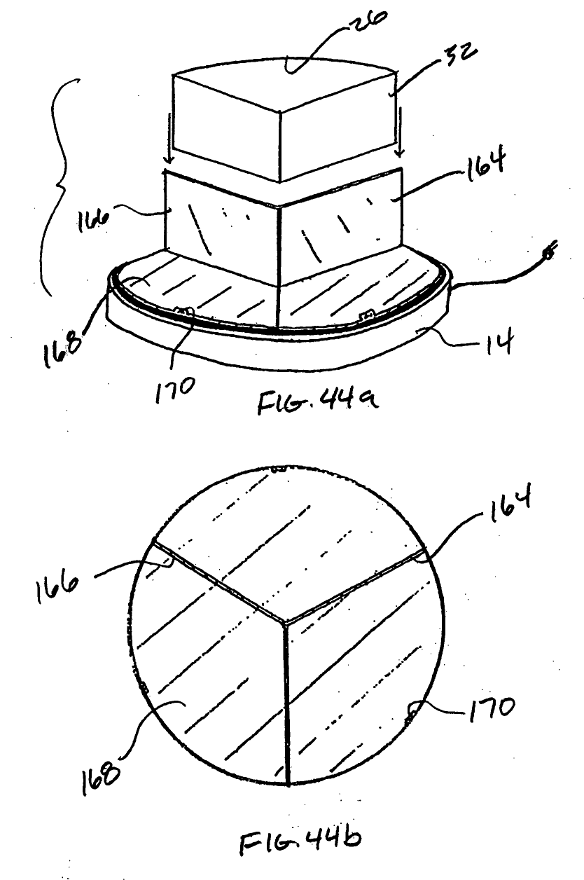

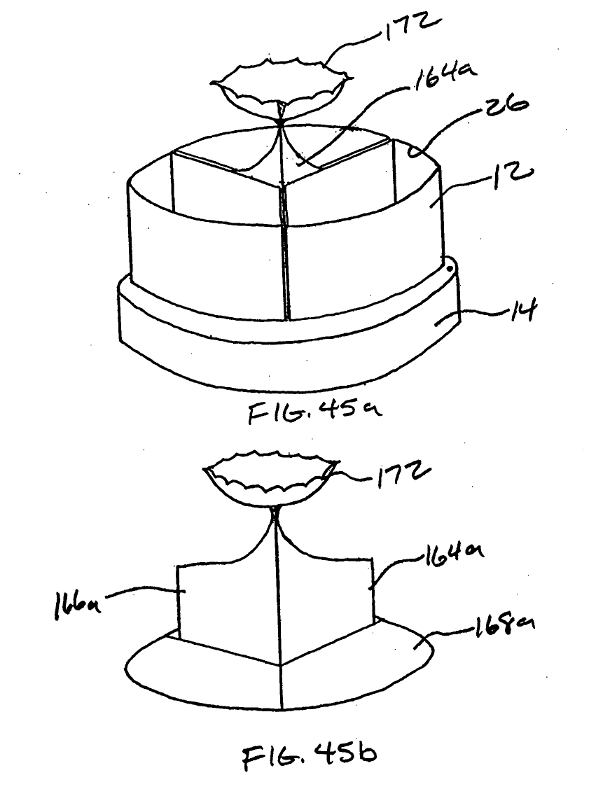

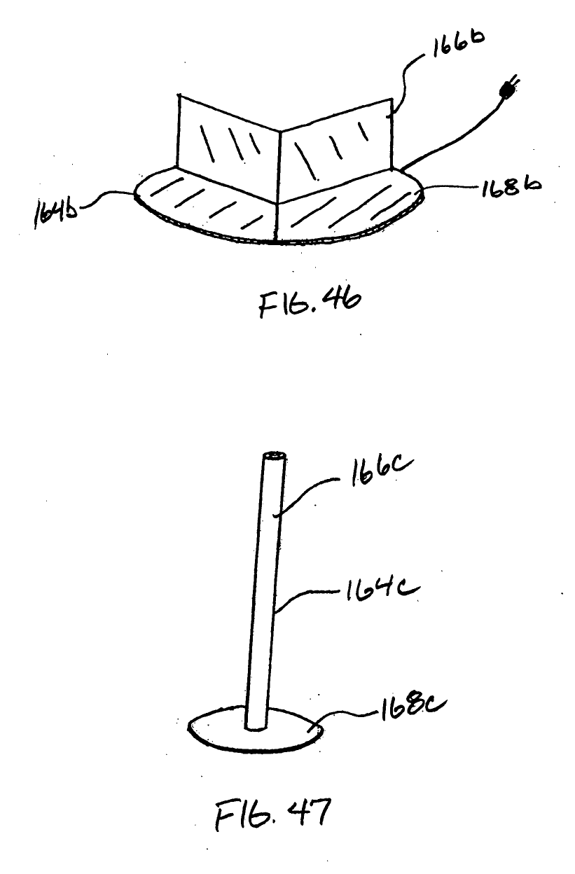

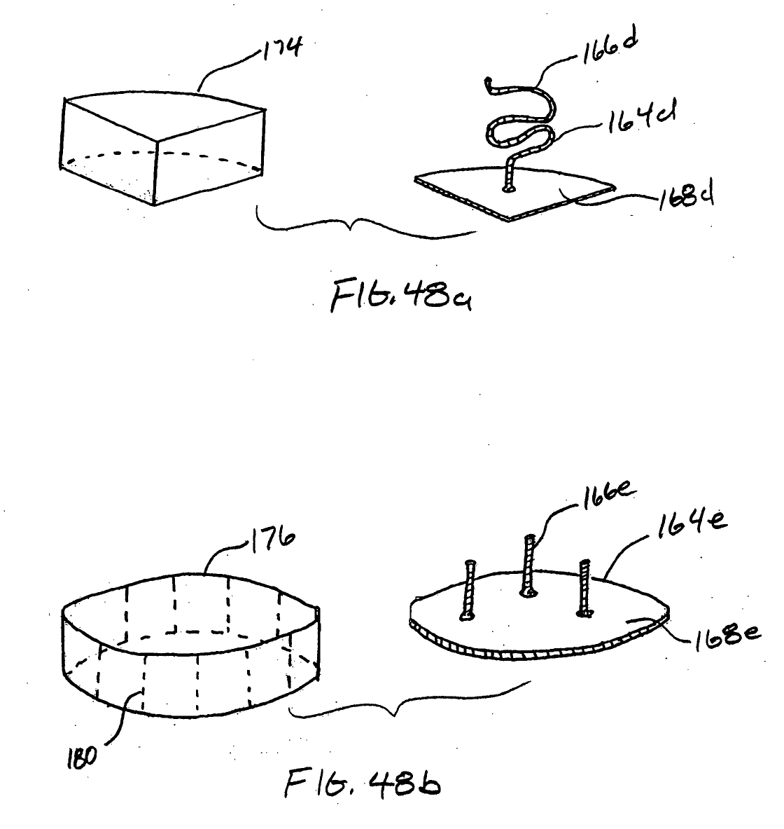

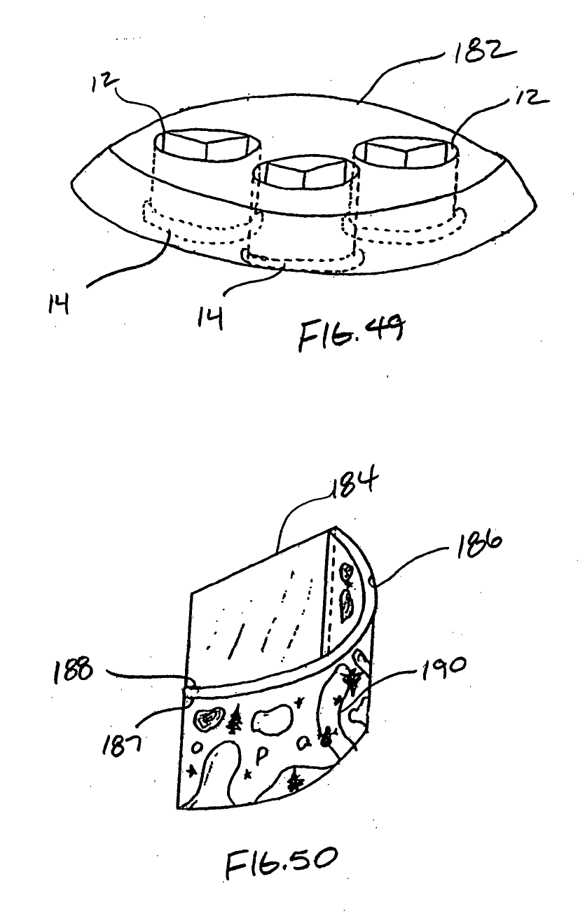

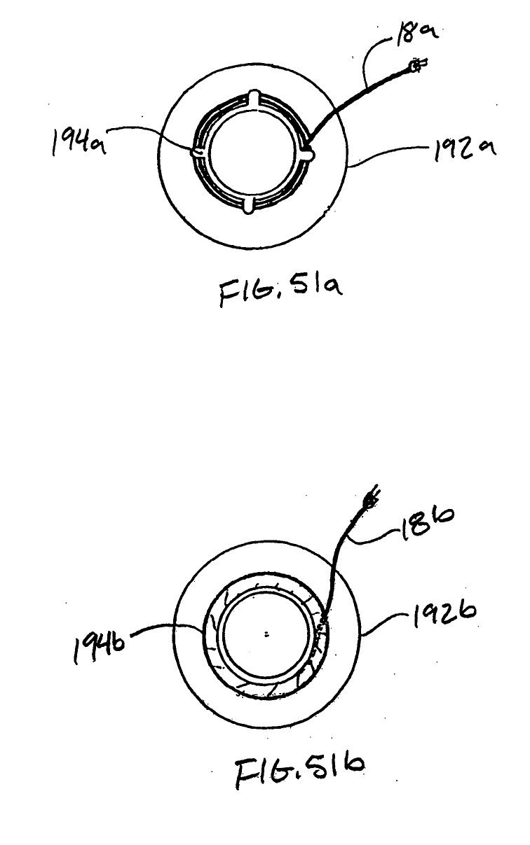

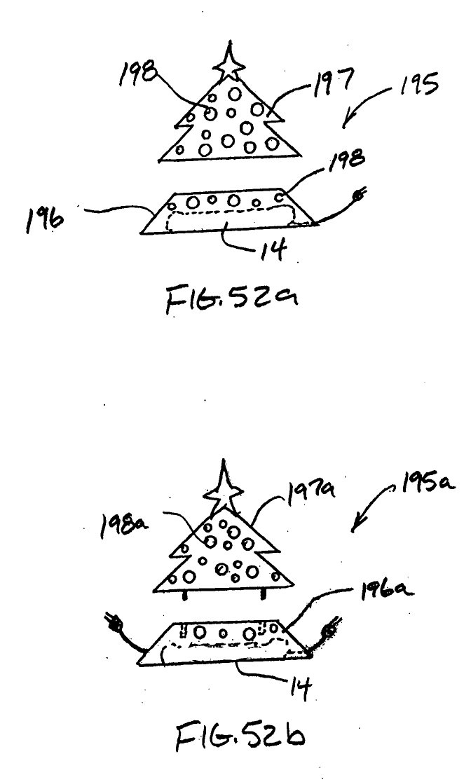

As shown in FIG. 26, another embodiment of a concentric container 82b is disclosed, in which an ornament 86 may be displayed. The ornament 86 may be configured in any desired shape for use with different themes or occasions. The ornament 86 may be fixed to the container 82b or one of the dividers 84b, or the ornament 86 may be removably connected to the container 82b or divider 84b. For example, the ornament 86 may include a projection 88 that may be received in an opening 90 formed on a divider 84b. The ornament 86 may be removed and replaced with a different ornament representing a different theme or occasion, for example.

As shown in FIG. 27, a further embodiment of the present disclosure is presented in which a container 82c is provided with non-uniform shaped compartments 85c. The compartments 85c may be discontinuous around a perimeter of the container 82c and/or the compartments 85c may be formed having the same or different heights. Also, portions 87 of the container 82c may be closed such that the compartments 85c do not encompass the entire volume of the container 82c. Moreover, the container 82c may be formed of multiple pieces or a single unitary piece. Accordingly, various different patterns or styles may be formed by the container 82c within the scope of the present disclosure.

Referring to FIGS. 28a-28c, it will be understood that containers 68p-68r may be formed in a one piece arrangement having a plurality of compartments 72p-72r that are configured to correspond to warmers 14p-14r. The embodiments shown in FIGS. 28a-28c are similar to the concept depicted in FIGS. 18a-18c discussed above, except that the containers 68p-68r may be formed as single piece units, rather than the multiple piece units depicted in FIGS. 18a-18c. It will be understood that the warmers 14p-14r may be formed with a ridge 22 defining a recess 24 as discussed above, such that the containers 68p-68r are sized to be received within the recess 24, or the warmers 14p-14r may be formed without the ridges 22.

Referring now to FIGS. 29a-29b, additional alternative embodiments of the present disclosure are shown in which lights 92, 92a are provided on a container 94, 94a. The lights 92 may be positioned on an exterior of the container 94, or the lights 92a may be positioned on an interior of the container 94a. It will also be understood that the lights 92, 92a may alternatively be configured to shine through both the interior and exterior of the container 94, 94a. The lights 92, 92a may be beneficial in creating a desired atmosphere, and may also be used to generate heat to warm a fragrance producing medium 28. It will be understood that the number, size and arrangement of the lights 92, 92a, may vary within the scope of the present disclosure. Moreover, any of a variety of containers and/or warmers disclosed herein may be used in combination with the lights 92, 92a.

Referring now to FIG. 30, an additional embodiment of the present disclosure is presented in which a snap-on lid 96 is shown in combination with the compartment 26. The lid 96 may be attached to the compartment 26 for preventing spillage of the fragrance producing medium 28. Accordingly, in one embodiment of the present disclosure, the lid 96 may provide a seal to hold the fragrance producing medium 28 within the compartment 26. Other embodiments of the lid 96 may not create an air-tight seal and some embodiments of the lid 96 may have a twist or threaded engagement. It will be understood that any type of attachment mechanism known in the art may be used to attach the lid 96 to the compartment 26. Also, it will be understood that the lid 96 may be configured to cover a single compartment 26, or a plurality of compartments 26 at a time, including the entire container 12.

Referring to FIGS. 31a and 31b, an additional embodiment of a container 78a is shown. The container 78a may be similar in concept to the container 78 in FIG. 23 discussed above, except that the container 78a may have a different shape and may include a lid 98. As shown in FIG. 31b, the container 78a may include a built in warmer 99 such that the container 78a may function as a unitary appliance. The lid 98 may be placed on the container 78a to cover the compartments 76a. The lid 98 may have openings or the lid 98 may be formed of a porous material that may allow a fragrance to pass therethrough. Alternatively, the lid may be used to prevent the fragrance producing medium 28 from spilling out of the compartments 76a, or to prevent a fragrance from being emitted from the compartments 76a. Similar to the other embodiments of the container discussed above, the container 78a may be formed in various different configurations and may have various different shapes and numbers of compartments 76a. Moreover, the container 78a and/or the lid 98 may include any variety of lights and/or decorations.

Referring now to FIGS. 32a-32e, an additional feature of the present disclosure is shown. As shown in FIGS. 32a-32e, containers 100a-100e may be provided that may be formed of a single part, similar to the containers of FIGS. 19-21 discussed above. However, the containers 100a-100e may not necessarily include a plurality of compartments, but rather the containers 100a-100e may include a single compartment 126. The containers 100a-100e may be formed to mate with warmers 114a-114e, in that a ridge 122a-122e may extend along at least a portion of the warmers 114a-114e above plate portions 116a-116e to form a recess 124a-124e for receiving a bottom portion 134a-134e of the containers 100a-100e. It will be understood that the ridge 122a-122e may be arranged in other configurations to provide a recess for receiving the containers 100a-100e such that the containers 100a-100e may be supported in a stable condition. This may be an improvement over prior art warmers and containers in that the prior art containers are not specifically sized to mate with a warmer such that the containers are less stable and less efficiently warmed. Moreover, the bottom portions 134a-134e of the present disclosure may be weighted to enhance the stability of the containers 100a-100e on the warmers 114a-114e. Some embodiments of the present disclosure may also include a flat surface 123a-123e on the bottom portions 134a-134e to enhance stability of the containers 100a-100e, and maximize the surface contact with the plate portions 116a-116e, to enhance the efficiency of heating the containers 100a-100e. It will also be understood that in alternative embodiments, ridges may be formed on the containers and recesses may be formed on the warmers to allow the containers to mate with the warmers.

Referring to FIGS. 33a and 33b, a further alternative embodiment of the present disclosure is presented. In the embodiment of FIGS. 33a and 33b, containers 140 may be provided to allow stackable containers 142 to be placed above the containers 140. The container 140 may have any of a variety of shapes and compartments, and may also include a heating mechanism. Electrical connectors 56a similar to those discussed above, may be provided in the containers 140 and the stackable container 142 to allow electrical power to be transmitted from the container 140 to the stackable containers 142. Accordingly, the container 140 may be used alone, or one or more stackable containers 142 may be placed above the container 140 to provide a plurality of compartments that may each be heated. In an alternative embodiment, heat conductors may be provided between the container 140 and the stackable containers 142 so that a fragrance producing medium 28 in the stackable containers 142 may be heated without necessarily transmitting electrical power from the container 140 to the stackable containers 142. In another embodiment of the present disclosure, openings may be provided in a bottom portion of the stackable containers 142 to allow a fragrance producing medium 28, such as wax, to melt and drip from an upper stackable container 142 to a lower stackable container 142, or to the container 140. In yet another embodiment of the present disclosure, the container 140 and the stackable containers 142 may be provided without a built in heating mechanism. Accordingly, the container 140 may be placed on a warmer 14 for heating the container 140. Similar to the other embodiments discussed above, the container 140 may be used alone, or any number of stackable containers 142 may be placed on the container 140 when the container 140 is placed on a warmer 14.

Turning now to FIG. 34, an additional embodiment of the present disclosure is shown in which a container 144 is depicted having a decorative divider 146. The decorative divider 146 may be fixed to the container 144, or the decorative divider 146 may be modular or removable. The decorative divider 146 may be configured to extend beyond the container 144 so as to be more visible, and the decorative divider 146 may include any ornamental appearance known to those skilled in the art, such as a tree as shown in FIG. 34. It will be understood that the decorative divider 146 may be used in combination with many of the other features described herein. Moreover, the decorative divider 146 may be solid or hollow and may include lights or ornamentation, such as sand, oil, bubbles, depictions, or any other variety of ornamentation, in the interior of the divider 146. Any variety of ornamentation or lights may also be provided on an exterior of the dividers 146.

Another feature is disclosed in FIGS. 35a-35c that may be useful in combination with many of the other features of the present disclosure. FIGS. 35a-35c disclose retainers 148a-148c that may be used to hold compartments 26 of a container 12 together. The retainers 148a-148c may be formed as a band or ring to encompass the container 12. The retainers 148a-148c may be formed with or without a floor, and the retainers 148a-148c may be formed of any suitable material, including a rigid material, a flexible material and/or an elastic material. The retainers 148a-148c may be formed in any shape to correspond to a container, such as round, rectangular, and triangular, for example. One embodiment of the retainer 148a may be formed as a ring without additional decorations. Alternatively, as shown in FIGS. 35b and 35c, additional embodiments may include any of a variety of ornamentation, such as flowers 150 or trees 152, for example. It will be understood that the ornamentation may be provided in any suitable arrangement or theme to enhance the appearance of the retainers 148a-148c. It will also be understood that the retainers may be used with single piece containers to add ornamentation without the need for holding multiple pieces together.

Another feature for attaching the compartments 26 of a container 12 may include connectors 154. Connectors 154 may be formed in any manner known in the art such as protrusions and openings, such that a protrusion on one of the compartments 26 may be received in an opening in an adjacent compartment 26 to thereby fasten the compartments together. One embodiment of the connector 154 may form a snap type connection to maintain the compartments 26 in alignment and position with respect to each other.

Alternatively, as shown in FIG. 37, a clip 156 may be provided to maintain the compartments 26 aligned and connected to each other. The clip 156 may include a groove 158 for receiving divider portions 38 of adjacent compartments 26. The clip 156 may be configured to be positioned at a central junction of a plurality of compartments, as shown in FIG. 37, or the clip 156 may be configured to be positioned at different locations along the divider portions 38 such that a plurality of clips 156 may be utilized to connect the compartments 26. The clip 156 may be formed in various different shapes and sizes, and the clip 156 may be made of any material suitable for withstanding heat generated by a warmer 14.

In one embodiment, the clip 156 may be formed of a metal material such that decorations having magnetic attachment mechanisms may be removably attached to the clip 156. As shown in FIG. 38, a decoration 160, such as a snowman for example, may be attached to the clip 156. It will be understood that the decoration 160 may be formed in any desired configuration. Moreover, the decoration may be fixedly attached to the clip 156 or removably attached to the clip 156 through a magnetic attachment mechanism or any other suitable attachment device known to those skilled in the art.

As shown in FIG. 39, a decorative retainer 148d may be used in combination with a decoration 160a to connect the compartments 26 of the container 12 as well as provide an ornamental theme for the container 12. An additional alternative embodiment decoration 160b is shown in FIG. 40 attached via a magnetic connection to a plate 162 on a clip 156. The clips 156 may be arranged in various different positions on the container 12, as shown in FIGS. 41 and 42. It will be understood that the clips 156 and decorations 160 may be used with many of the embodiments of the container, including one part embodiments such as those disclosed in FIGS. 19-21. Accordingly, the clips 156 and decorations 160 may be used to provide ornamentation to the containers without connecting compartments. It will also be understood that the clips 156 and decorations 160 may be used to enhance the safety and/or appearance of divided multi-part containers, though the clips 156 and decorations 160 may not be necessary in certain situations.

A further alternative embodiment clip 156a is shown in FIGS. 43a and 43b. The clip 156a may be formed integrally with the sidewall 32 of the container 12, or the clip 156a may be separate from the container 12. The clip 156a may be formed as a fold defining a groove 158a for receiving the divider portion 38 of an adjacent compartment 26. The embodiment of the clip 156a in FIGS. 43a and 43b may or may not include a plate 162 for attaching a decoration.

An additional feature of the present disclosure is shown in FIGS. 44a and 44b. A heat conductor 164 may be provided to transfer heat to at least a portion of the sidewall 32 of a compartment 26 of a container 12. The heat conductor 164 may include upright portions 166 and a lateral portion 168, though some embodiments may be formed without the lateral portion 168. It will be understood that the heat conductor 164 may be formed of any suitable material for transferring heat from a warmer 14 to the compartment 26. The heat conductor 164 may be attached to the warmer using an attachment mechanism 170, such as tabs or fasteners or any other attachment device known to those skilled in the art. Other embodiments of the heat conductor 164 may not be attached to the warmer 14. As the warmer 14 generates heat, the heat may be transferred from the warmer 14 through the heat conductor 164 to the sidewalls 32 of the compartments 26 so that a larger surface area of the compartments 26 may be heated. This may accelerate heating of the fragrance producing medium 28 so that an aroma is produced faster or in greater quantities. It will be appreciated that the heat conductor 164 may be formed in various different configurations compatible with the various embodiments discussed herein, and the heat conductor 164 may be placed on the inside or outside of the container compartments 26. Moreover, as shown in FIGS. 45a and 45b, a receptacle 172 may be positioned on the heat conductor 164a for receiving a fragrance producing medium 28 or any other item or decoration. One embodiment of the receptacle 172 may be formed of a heat conducting material similar to the material in the heat conductor 164a to transfer heat though the receptacle 172. Alternatively, the receptacle 172 may be formed of a material providing insulation characteristics such that the receptacle may be configured to receive items that may not be intended to be heated.

Referring now to FIG. 46, an additional embodiment conductor 164b is shown. As previously discussed, the presently disclosed embodiments illustrated herein are merely exemplary of the possible embodiments of the disclosure, including that illustrated in FIG. 46. The conductor 164b may be formed such that the lateral portion 168b and/or the upright portion 166b may be formed with a lighting mechanism to function as an illumination device for a container. The lighting mechanism may be formed of any variety of light emitting devices known in the art, such as fiber optics, fluorescent, neon, or indiglo lighting, or LED technology, for example. It will be understood that any portion of the conductor 164b may have a lighting mechanism, or the entire conductor 164b may include the lighting mechanism. Moreover, the conductor 164b may be formed in any desired size and configuration. The lighting mechanism may also assist in generating heat, in some embodiments, to warm the container.

Referring now to FIG. 47, an additional embodiment conductor 164c is shown. The conductor 164c may be placed inside a container to conduct heat through a fragrance producing media more rapidly. Other embodiments of the conductor 164c may be placed outside the container. It will be understood that the conductor 164c may be used with any of the containers disclosed herein, including single compartment, multi compartment, one piece or multiple piece containers, and that any number of conductors 164c may be placed in the compartments. The conductor 164c may be provided in combination with a container, or the conductor 164c may be provided as a separate accessory. The conductor 164c may be provided with a base or lateral portion 168c and an upright portion 166c. The conductor 164c may be formed of any variety of heat conductive materials, and may be formed in any height, size or shape. Also, the conductor 164c may be formed such that at least a portion of the conductor 164c is hollow, or the entire conductor 164c may be solid.

Referring to FIGS. 48a and 48b, conductors 164d, 164e may be provided with base or lateral portions 168d, 168e that may be shaped to correspond to the shape of a compartment. This may enhance the heat transfer efficiency and improve the stability of the conductors 164d, 164e. As shown in FIG. 48a, the compartment 174 may be part of a multi compartment container. The conductor 164d may have an upright portion 166d that may be non-straight so as to have an increased length to provide increased surface area for transferring heat to a fragrance producing medium. Alternatively, as shown in FIG. 48b, a single compartment container 176 may receive the conductor 164e, and the conductor 164e may be provided with a plurality of upright portions 166e. It will be understood that any combination of different quantities and configurations of upright portions 166d, 166e may be provided for use in either single compartment or multi compartment containers.

It will also be understood, as shown in FIG. 48b, that embodiments of the present disclosure may include compartments or containers 176 that may include heat conductors 180 (shown in dashed lines) either embedded within the interior of the sidewall of the container 176, or positioned on the side of the sidewall of the container 176. The conductors 180 may be in the form of wires that are configured to transfer heat to the container 176 to heat a fragrance producing medium. It will be understood that the conductors 180 may be used alone or in combination with many of the other features disclosed herein, including single compartment, multiple compartment, single piece, and multi piece containers.

Referring to FIG. 49, an alternative embodiment cover 182 is shown in which a plurality of containers 12 may be received within the cover 182. The cover 182 may also be configured to receive a plurality of warmers 14 within the cover 182. It will be understood that the size, shape and configuration of the cover 182 may be formed in any desirable manner for receiving any number of containers 12 and warmers 14. Moreover, the cover 182 may be configured as an appliance having one or more warmers integrally formed within the cover 182, and many of the other features disclosed herein may be used in combination with the cover 182.

An additional feature of the present disclosure is presented in FIG. 50, which shows a compartment 184 having a hollow sidewall portion 186. It will be understood that the compartment 184 may be formed in any size and shape, and the compartment 184 may be one of several pieces of a container as discussed above, or the compartment 184 may be a single piece container. The hollow portion 186 may be formed on any portion of or the entire compartment 184. The hollow portion 186 may be formed by an exterior wall 187 and an interior wall 188 to define a hollow space therebetween. The hollow portion 186 may be configured to receive ornamentation 190, such as a heat reactant media, including oils, bubbles, and colorants, for example. Decorative items may be suspended in the oil to provide a changing display. Moreover, it will be understood that any variety of ornamentation 190 may be placed within the hollow portion 186, including liquids, lights, or colored particulate matter, such as sand, within the scope of the present disclosure, and the ornamentation may be movable or fixed within the hollow portion 186.

Referring now to FIGS. 51a and 51b, additional features of the present disclosure are shown. As is shown in the bottom view of FIG. 51a, a cover 192a may be provided to include a winding means 194a for winding an electrical cord 18a. The winding means 194a may include any variety of channels, projections or tabs for receiving the cord 18a, such that the cord 18a may be manually wound into a coil around the cover 192a. Alternatively, as shown in FIG. 51b, an automatic winding means 194b may be provided for receiving the cord 18b. The automatic winding means 194b may include a spring tensioned device that allows the cord 18b to be wound into a coil automatically such that any portion of the cord 18b that is not in use may be stored or hidden from view. It will be understood that any suitable winding mechanism known in the art may be used to form the winding means 194b within the principles of the present disclosure. Moreover, the winding means 194a, 194b may be utilized in combination with many of the other features disclosed herein.

Referring now to FIGS. 52a and 52b, additional embodiments of the present disclosure are shown. It will be appreciated that the embodiments of the disclosure illustrated in FIGS. 52a and 52b contain many of the same structures represented in FIGS. 14a-14d and only the new or different structures will be explained to most succinctly explain the additional advantages which come with the embodiments of the disclosure illustrated in FIGS. 52a and 52b.

As shown in FIG. 52a, a cover 195 is disclosed having a base 196 and a lid 197. The base 196 may be configured to receive a warmer 14 and/or a container. The base 196 and/or the lid 197 may include openings 198 for allowing light from a candle or warmer 14 within the cover 195 to be visible. Also, the openings 198 may allow an aroma produced within the cover 195 to pass outside of the cover 195 more freely. It will be understood that the size, location, quantity and configuration of the openings 198 may vary as desired within the scope of the present disclosure.

As shown in FIG. 52b, a cover 195a may be provided as a separate appliance having its own light source. The light source may be provided integral with the cover 195a such that a candle or separate light on a warmer is not needed to produce light within the cover 195a. Other embodiments of the cover 195a may include lights on the exterior surface of the cover 195a. In one embodiment, the lid 197a may be connected to the base 196a with electrical connectors 199 such that the lid 197a may be electrically coupled to the base 196a to power lights in the lid 197a and/or the base 196a.

It will be understood that the containers described in the above embodiments may be formed of any suitable material known in the art that may be capable of withstanding heat applied by a warmer. For example, the containers may be formed of a glass, ceramic, metal or plastic material having the desired heat conductivity, strength, and durability characteristics. One embodiment of the container may be formed of a transparent glass material that may allow light and/or the color of the fragrance producing medium 28 to be viewed through the container. The container may also be substantially rigid to support the fragrance producing medium 28 in place even under increased temperature conditions. However, it will be understood that the container may be formed of other materials having other characteristics within the scope of the present disclosure.

It will be appreciated that the structure and apparatus disclosed herein is merely one example of a means for containing a plurality of fragrance producing media, and it should be appreciated that any structure, apparatus or system for containing a plurality of fragrance producing media which performs functions the same as, or equivalent to, those disclosed herein are intended to fall within the scope of a means for containing a plurality of fragrance producing media, including those structures, apparatus or systems for containing a plurality of fragrance producing media which are presently known, or which may become available in the future. Anything which functions the same as, or equivalently to, a means for containing a plurality of fragrance producing media falls within the scope of this element.

It will be appreciated that the structure and apparatus disclosed herein is merely one example of a means for heating a fragrance producing media, and it should be appreciated that any structure, apparatus or system for heating a fragrance producing media which performs functions the same as, or equivalent to, those disclosed herein are intended to fall within the scope of a means for heating a fragrance producing media, including those structures, apparatus or systems for heating a fragrance producing media which are presently known, or which may become available in the future. Anything which functions the same as, or equivalently to, a means for heating a fragrance producing media falls within the scope of this element.

It will be appreciated that the structure and apparatus disclosed herein is merely one example of a means for mateingly engaging, and it should be appreciated that any structure, apparatus or system for mateingly engaging which performs functions the same as, or equivalent to, those disclosed herein are intended to fall within the scope of a means for mateingly engaging, including those structures, apparatus or systems for mateingly engaging which are presently known, or which may become available in the future. Anything which functions the same as, or equivalently to, a means for mateingly engaging falls within the scope of this element.

In accordance with the features and combinations described above, a useful method of producing an aroma includes the steps of:
(a) separating a container into a plurality of compartments that are moveable with respect to each other;
(b) placing a fragrance producing medium in each of the plurality of compartments;
(c) placing the container on a warmer device; and
(d) warming the fragrance producing medium.

In accordance with the features and combinations described above, another useful method of producing an aroma includes the steps of:
(a) placing a first fragrance producing medium in a first compartment;
(b) placing a second fragrance producing medium in a second compartment;
(c) mating the first compartment and the second compartment; and
(d) warming the first fragrance producing medium and the second fragrance producing medium to produce the aroma.

Those having ordinary skill in the relevant art will appreciate the advantages provide by the features of the present disclosure. For example, it is a feature of the present disclosure to provide an apparatus for producing an aroma which is simple in design, manufacture and use. Another feature of the present disclosure is to provide such an apparatus for producing an aroma that allows a custom, blended aroma to be created. It is a further feature of the present disclosure, in accordance with one aspect thereof, to provide an apparatus for producing an aroma, in which a container is configured to mate in a stable condition with a warmer. It is an additional feature of the present disclosure to provide an apparatus for producing an aroma in which the apparatus has a pleasing aesthetic appearance that can be used for various different themes and occasions. It is a further feature of the present disclosure to provide an apparatus for producing an aroma that allows a plurality of fragrances to be produced in a clean manner.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. An apparatus for use in producing an aroma, said apparatus comprising:
a container having a plurality of compartments, each of said compartments comprising a bottom portion and a sidewall portion such that each of said plurality of compartments is configured to receive a fragrance producing medium thereinto; and
a means for heating said container;
wherein each bottom portion of said plurality of compartments is configured to be received in a recess in said means for heating said container, and wherein said plurality of compartments are moveable with respect to each other and mateable to form said container such that when said container is placed in said recess in said means for heating, said fragrance producing medium can be heated to produce said aroma.

2. The apparatus of claim 1, further comprising a fragrance producing medium disposed in said plurality of compartments.

3. The apparatus of claim 2, wherein said fragrance producing medium is selected from the group consisting of scented wax, potpourri, solid fragrance, liquid fragrance, oils, and gels.

4. The apparatus of claim 1, wherein each of said plurality of compartments includes a different scented fragrance producing medium therein.

5. The apparatus of claim 1, wherein said means for heating said container comprises a heating surface.

6. The apparatus of claim 5, further comprising a ridge circumscribing said heating surface for defining said recess.

7. The apparatus of claim 1, wherein each of said plurality of compartments comprises a divider portion.

8. The apparatus of claim 7, wherein said divider portion is formed as part of said sidewall of said plurality of compartments.

9. The apparatus of claim 1, wherein said sidewall extends in a substantially perpendicular orientation with respect to said bottom portion.

10. The apparatus of claim 1, wherein an exterior portion of said sidewall extends in a substantially convex configuration.

11. The apparatus of claim 1, wherein an exterior portion of said sidewall extends in a substantially concave configuration.

12. The apparatus of claim 1, wherein said plurality of compartments comprises three compartments.

13. The apparatus of claim 1, wherein said plurality of compartments comprises two compartments.

14. The apparatus of claim 1, wherein said plurality of compartments comprises four compartments.

15. The apparatus of claim 1, wherein said bottom portion has a thicker dimension than said sidewall portion.

16. The apparatus of claim 1, wherein said bottom portion is formed to be heavier than said sidewall portion.

17. The apparatus of claim 1, further comprising a cover for covering said means for heating said container.

18. The apparatus of claim 17, wherein said cover comprises a plurality of sections.

19. The apparatus of claim 18, wherein said plurality of sections are electrically coupled together.

20. The apparatus of claim 17, wherein said cover is formed as one piece unit.

21. The apparatus of claim 1, wherein said apparatus further comprises a bowl for receiving said container.

22. The apparatus of claim 21, further comprising a lid for covering said bowl.

23. The apparatus of claim 22, wherein said lid comprises one or more openings.

24. The apparatus of claim 23, wherein said lid further comprises one or more stops for covering said one or more openings.

25. The apparatus of claim 24, wherein said one or more stops are configured to operate in unison to open or close said one or more openings at the same time.

26. The apparatus of claim 24, wherein said one or more stops are independently adjustable to open or close the one or more openings independently.

27. The apparatus of claim 1, wherein said plurality of compartments are arranged in a concentric configuration.

28. The apparatus of claim 1, wherein said plurality of compartments have different heights.

29. The apparatus of claim 1, wherein said container further comprises an ornament.

30. The apparatus of claim 1, wherein said plurality of compartments each have substantially identical shapes.

31. The apparatus of claim 1, wherein said plurality of compartments have substantially different shapes.

32. The apparatus of claim 1, wherein said container comprises one or more lights.

33. The apparatus of claim 32, wherein said one or more lights are positioned on an exterior of the container.

34. The apparatus of claim 32, wherein said one or more lights are positioned on an interior of the container.

35. The apparatus of claim 1, wherein each of said plurality of compartments comprise a snap-on lid.

36. The apparatus of claim 1, further comprising a retainer for holding the plurality of compartments together.

37. The apparatus of claim 36, wherein the retainer comprises a band encompassing the container.

38. The apparatus of claim 1, further comprising a clip for connecting the plurality of compartments together.

39. The apparatus of claim 38, wherein said clip comprises a groove for receiving a divider portion of the container.

40. The apparatus of claim 38, wherein said clip is formed integrally with the sidewall.

41. The apparatus of claim 38, wherein said clip comprises a plate for magnetically connecting a decoration.

42. The apparatus of claim 1, further comprising a connector for connecting the plurality of compartments together, wherein said connector comprises a protrusion on one of the plurality of compartments and an opening for receiving the protrusion on another of the plurality of compartments.

43. The apparatus of claim 1, further comprising a conductor for transferring heat to at least a portion of the sidewall.

44. The apparatus of claim 43, wherein said conductor comprises a receptacle for receiving a fragrance producing medium.

45. An apparatus for producing an aroma, said apparatus comprising:
a container having a first compartment and a second compartment, said first compartment and said second compartment being mateable with each other and movable with respect to each other;
a first fragrance producing medium in said first compartment; and
a second fragrance producing medium in said second compartment;
wherein said container is configured to be heated to produce said aroma of said first fragrance producing medium and said second fragrance producing medium.

46. The apparatus of claim 45, wherein at least one of said first fragrance producing medium and said second fragrance producing medium comprises wax.

47. The apparatus of claim 45, wherein at least one of said first fragrance producing medium and said second fragrance producing medium comprises a solid fragrance material.

48. The apparatus of claim 45, wherein at least one of said first fragrance producing medium and said second fragrance producing medium comprises a liquid fragrance material.

49. The apparatus of claim 45, wherein at least one of said first fragrance producing medium and said second fragrance producing medium comprises a scented oil material.

50. The apparatus of claim 45, wherein at least one of said first fragrance producing medium and said second fragrance producing medium comprises an aromatic gel material.

51. The apparatus of claim 45, wherein said first fragrance producing medium produces a different fragrance than said second fragrance producing medium.

52. The apparatus of claim 45, further comprising a means for heating said container.

53. The apparatus of claim 52, wherein said means for heating said container comprises a heating surface and a ridge circumscribing said heating surface for defining a recess.

54. The apparatus of claim 45, wherein said first compartment and said second compartment each comprise a bottom portion and a sidewall portion, said bottom portion being configured to be received in a recess in a means for heating said container.

55. The apparatus of claim 45, further comprising a third compartment and a third fragrance producing medium.

56. An apparatus for producing an aroma, said apparatus comprising:
a container having a plurality of compartments for receiving a fragrance producing medium; and
a warmer for heating said container;
wherein said warmer has a recess and said container is receivable in said recess such that said warmer is configured to heat said container and said fragrance producing medium.

57. The apparatus of claim 56, wherein said warmer comprises a heating surface.

58. The apparatus of claim 57, further comprising a ridge circumscribing said heating surface for defining said recess.

59. The apparatus of claim 56, wherein said warmer comprises a plate that generates heat using electrical power.

60. The apparatus of claim 56, wherein said plurality of compartments are moveable with respect to each other.

61. The apparatus of claim 56, wherein each of said compartments comprise a bottom portion and a sidewall portion such that said bottom portion is configured to be received in said recess.

62. The apparatus of claim 56, further comprising a fragrance producing medium.

63. The apparatus of claim 62, wherein said fragrance producing medium is selected from the group consisting of scented wax, potpourri, solid fragrance, liquid fragrance, oils, and gels.

64. The apparatus of claim 56, wherein each of said plurality of compartments includes a different scented fragrance producing medium therein.

65. An apparatus for producing an aroma, said apparatus comprising:
a container for receiving a fragrance producing medium, said container comprising a bottom portion defining a perimeter, said container further comprising a sidewall, at least a portion of said sidewall extending laterally beyond said perimeter of said bottom portion; and
a warmer for generating heat to warm said container and said fragrance producing medium, said warmer comprising a ridge defining a recess configured to receive said bottom of said container therein;
wherein when said bottom of said container is received in said recess, said sidewall of said container resides directly above at least a portion of said ridge.

66. The apparatus of claim 65, wherein said container covers at least a portion of said ridge.

67. The apparatus of claim 65, wherein said container comprises a plurality of compartments.

68. The apparatus of claim 67, wherein said plurality of compartments are moveable with respect to each other.

69. The apparatus of claim 65, further comprising said fragrance producing medium disposed in said container.

70. The apparatus of claim 69, wherein said fragrance producing medium is selected from the group consisting of scented wax, potpourri, solid fragrance, liquid fragrance, oils, and gels.

71. The apparatus of claim 65, wherein each of said plurality of compartments includes a different scented fragrance producing medium therein.

72. The apparatus of claim 65, wherein said bottom portion has a thicker dimension than said sidewall portion.

73. The apparatus of claim 65, wherein said bottom portion is formed to be heavier than said sidewall portion.

74. An apparatus for producing an aroma, said apparatus comprising:
a container having a divider removably receivable in said container for separating said container into a plurality of compartments, said plurality of compartments configured for receiving a fragrance producing medium;
a warmer for generating heat to warm said container and said fragrance producing medium;
wherein said divider is configured to allow said fragrance producing medium to be separated such that a different fragrance producing medium can be placed in each of said plurality of compartments to produce said aroma when said container is warmed.

75. The apparatus of claim 74, further comprising said fragrance producing medium.

76. The apparatus of claim 75, wherein said fragrance producing medium is selected from the group consisting of scented wax, potpourri, solid fragrance, liquid fragrance, oils, and gels.

77. The apparatus of claim 74, wherein each of said plurality of compartments includes a different scented fragrance producing medium therein.

78. The apparatus of claim 74, wherein said warmer comprises a heating surface.

79. The apparatus of claim 78, further comprising a ridge circumscribing said heating surface for defining a recess.

80. An apparatus for producing an aroma, said apparatus comprising:
a container having a plurality of compartments that are moveable with respect to each other and mateable with each other, said plurality of compartments configured for receiving a fragrance producing medium;
a warmer for generating heat to warm said container and said fragrance producing medium;
wherein said plurality of compartments are configured to allow said fragrance producing medium to be separated such that a different fragrance producing medium can be placed in each of said plurality of compartments to produce said aroma when said container is warmed.

81. The apparatus of claim 80, wherein each of said plurality of compartments comprises a bottom portion and a sidewall portion.

82. The apparatus of claim 80, further comprising said fragrance producing medium.

83. The apparatus of claim 82, wherein said fragrance producing medium is selected from the group consisting of scented wax, potpourri, solid fragrance, liquid fragrance, oils, and gels.

84. The apparatus of claim 80, wherein each of said plurality of compartments includes a different scented fragrance producing medium therein.

85. The apparatus of claim 80, wherein each of said plurality of compartments includes said fragrance producing medium therein, and wherein said fragrance producing medium in each of said plurality of compartments produces substantially the same fragrance.

86. The apparatus of claim 80, wherein said warmer comprises a heating surface.

87. The apparatus of claim 86, further comprising a ridge circumscribing said heating surface for defining a recess.

88. An apparatus for producing an aroma, said apparatus comprising:
a container having a plurality of compartments configured for receiving a fragrance producing medium, said container further comprising a cover defining an interior space;
a warmer for generating heat to warm said plurality of compartments and said fragrance producing medium;
wherein said warmer is receivable in said interior space to cover said warmer when said plurality of compartments are being warmed by said warmer.

89. The apparatus of claim 88, wherein said plurality of compartments are moveable with respect to each other.

90. The apparatus of claim 88, further comprising said fragrance producing medium.

91. The apparatus of claim 90, wherein said fragrance producing medium is selected from the group consisting of scented wax, potpourri, solid fragrance, liquid fragrance, oils, and gels.

92. The apparatus of claim 88, wherein each of said plurality of compartments includes a different scented fragrance producing medium therein.

93. The apparatus of claim 88, wherein said warmer comprises a heating surface.

94. The apparatus of claim 93, further comprising a ridge circumscribing said heating surface for defining a recess.

95. The apparatus of claim 94, wherein each of said plurality of compartments comprises a bottom portion and a sidewall portion, wherein said bottom portion is configured to be received in said recess.

96. The apparatus of claim 88, wherein said cover comprises a plurality of sections.

97. The apparatus of claim 96, wherein said plurality of sections are electrically coupled together.

98. A method for producing an aroma, said method comprising the steps of:
(a) separating a container into a plurality of compartments that are moveable with respect to each other;
(b) placing a fragrance producing medium in each of said plurality of compartments;
(c) placing said container on a warmer device; and
(d) warming said fragrance producing medium.

99. The method of claim 98, wherein step (b) comprises placing a different scented fragrance producing medium in each of said plurality of compartments.

100. The method of claim 98, wherein step (b) comprises selecting said fragrance producing medium from the group consisting of scented wax, potpourri, solid fragrance, liquid fragrance, oils, and gels.

101. The method of claim 98, further comprising connecting said plurality of compartments together.

102. The method of claim 98, further comprising mating a bottom portion of said plurality of compartments in a recess in said warmer.

103. A method for producing an aroma, said method comprising the steps of:
(a) placing a first fragrance producing medium in a first compartment;
(b) placing a second fragrance producing medium in a second compartment;
(c) mating said first compartment and said second compartment; and
(d) warming said first fragrance producing medium and said second fragrance producing medium to produce said aroma.

104. The method of claim 103, wherein said first fragrance producing medium has a different scent than said second fragrance producing medium.

105. The method of claim 103, wherein said first fragrance producing medium and said second fragrance producing medium are selected from the group consisting of scented wax, potpourri, solid fragrance, liquid fragrance, oils, and gels.

106. The method of claim 103, further comprising connecting said first compartment and said second compartment together.

107. The method of claim 103, wherein step (d) comprises the step of placing said first compartment and said second compartment on a warmer.

108. The method of claim 107, further comprising mating a bottom portion of said first compartment and a bottom portion of said second compartment in a recess in said warmer.

109. An apparatus for producing an aroma, said apparatus comprising:
means for containing a plurality of fragrance producing media; and
means for heating said fragrance producing media;
wherein at least one of said means for containing and said means for heating comprises a means for mateingly engaging with the other of said means for containing and said means for heating.

110. The apparatus of claim 109, wherein said means for containing comprises a container having a plurality of compartments.

111. The apparatus of claim 110, wherein each of said plurality of compartments includes a different scented fragrance producing medium therein.

112. The apparatus of claim 110, wherein said plurality of compartments are moveable with respect to each other.

113. The apparatus of claim 109, wherein said means for heating comprises a warmer having a heating surface.

114. The apparatus of claim 109, wherein said means for mateingly engaging comprises a recess on said means for heating configured for receiving a bottom portion of said means for containing.

115. The apparatus of claim 114, wherein said recess is defined by a rim at least partially circumscribing a heating surface.

116. The apparatus of claim 109, further comprising said fragrance producing medium.

117. The apparatus of claim 116, wherein said fragrance producing medium is selected from the group consisting of scented wax, potpourri, solid fragrance, liquid fragrance, oils, and gels.

118. A container for use with a warmer for producing an aroma, said warmer comprising a portion configured for generating heat, and a ridge defining a recess for receiving said container, said container comprising:
a plurality of compartments configured for receiving a fragrance producing medium, each of said plurality of compartments comprising a bottom portion and a sidewall portion, said plurality of compartments being moveable with respect to each other and mateable with each other;
wherein said bottom portion of each of said plurality of compartments is configured to be received in said recess in said warmer for heating said container and said fragrance producing medium to thereby produce said aroma.

119. An apparatus for use in producing an aroma, said apparatus comprising:
a container having a plurality of compartments, each of said compartments comprising a bottom portion extending in a substantially lateral orientation, and a sidewall portion extending in a substantially upward orientation;
a fragrance producing medium disposed in each of the plurality of compartments; and
a means for heating said container, said means for heating said container comprising a heating surface and a ridge circumscribing said heating surface defining a cylindrical recess;
wherein each bottom portion of said plurality of compartments is configured to be received in said recess;
wherein said plurality of compartments are moveable with respect to each other and mateable to form said container;
wherein said fragrance producing medium is selected from the group consisting of scented wax, potpourri, solid fragrance, liquid fragrance, oils, and gels;
wherein each of said plurality of compartments includes a different scented fragrance producing medium therein;
wherein each of said plurality of compartments comprises a divider portion that is mateable with another of said plurality of compartments;
wherein each of said divider portions is fixedly attached to one of said plurality of compartments;
wherein said plurality of compartments have substantially identical shapes formed as a segment of a cylinder;
wherein said plurality of compartments mate together to form a cylindrical shape;
wherein each of said bottom portions of said plurality of compartments comprises a flat surface for contacting said heating surface;
wherein when said container is placed in said recess on said heating surface of said means for heating, said fragrance producing medium is heated to produce said aroma as a blend of said different scented fragrance producing media.

120. The apparatus of claim 119, wherein said container is formed of a substantially transparent glass material;
wherein said sidewall of each of said plurality of compartments extends in a direction substantially perpendicular to said bottom portion;
wherein said plurality of compartments comprises three compartments; and
wherein a thickness of said bottom portion is greater than a thickness of said sidewall.

121. The apparatus of claim 119, wherein said container is formed of a substantially transparent glass material;
wherein each of said bottom portions of said plurality of compartments defines a perimeter;
wherein at least a portion of said sidewall extends laterally beyond said perimeter to cover said ridge on said means for heating said container;
wherein said bottom portion of each of said plurality of compartments is formed to be heavier than said sidewall; and
wherein said plurality of compartments are held together with a retainer, said retainer comprising a band encompassing said container.

122. The apparatus of claim 119, wherein said container is formed of a substantially transparent glass material;
wherein one or more lights are disposed on said sidewall;
wherein said plurality of compartments are connected together with one or more clips; and
wherein decorations are removably attachable to said one or more clips.

123. An apparatus for use in producing an aroma, said apparatus comprising:
a container having a plurality of compartments, each of said compartments comprising a bottom portion and a sidewall portion such that each of said plurality of compartments is configured to receive a fragrance producing medium thereinto;
wherein each bottom portion of said plurality of compartments is configured to be received in a recess in a means for heating said container, and wherein said plurality of compartments are moveable with respect to each other and mateable to form said container such that when said container is placed in said recess in said means for heating, said fragrance producing medium can be heated to produce said aroma.

124. An apparatus for producing an aroma, said apparatus comprising:
a container having a first compartment and a second compartment, said first compartment and said second compartment being mateable with each other and movable with respect to each other;
a first fragrance producing medium in said first compartment;
a second fragrance producing medium in said second compartment; and
a means for heating said container;
wherein said container is configured to be heated to produce said aroma of said first fragrance producing medium and said second fragrance producing medium.

125. An apparatus for producing an aroma, said apparatus comprising:
a container having a first compartment and a second compartment, said first compartment and said second compartment being mateable with each other and movable with respect to each other;
a first fragrance producing medium in said first compartment;
a second fragrance producing medium in said second compartment; and
a means for heating said container, said means for heating said container comprising a bump defining a recess for receiving said container;
wherein said container is configured to be heated to produce said aroma of said first fragrance producing medium and said second fragrance producing medium.

126. An apparatus for producing an aroma, said apparatus comprising:
a container having a first compartment and a second compartment, said first compartment and said second compartment being mateable with each other and movable with respect to each other;
a first fragrance producing medium in said first compartment; and
a second fragrance producing medium in said second compartment;
wherein said first fragrance producing medium produces a different fragrance than said second fragrance producing medium; and
wherein said container is configured to be heated to produce said aroma of said first fragrance producing medium and said second fragrance producing medium.

127. An apparatus for producing an aroma, said apparatus comprising:
a container for receiving a fragrance producing medium, said container comprising a bottom portion defining a perimeter, said container further comprising a sidewall; and
a warmer for generating heat to warm said container and said fragrance producing medium, said warmer comprising a ridge defining a recess configured to receive said bottom of said container therein;
wherein when said bottom of said container is received in said recess, said sidewall of said container resides directly above at least a portion of said ridge, and at least a portion of said sidewall extends laterally beyond said ridge.

128. The apparatus of claim 43, wherein said conductor comprises a lighting mechanism.

129. The apparatus of claim 1, further comprising a conductor in said container for conducting heat through said fragrance producing medium.

130. The apparatus of claim 129, wherein said conductor has a base portion configured to correspond to at least one of said plurality of compartments.

131. The apparatus of claim 129, wherein said conductor comprises a plurality of upright portions.

132. The apparatus of claim 129, wherein said conductor is formed in said sidewall portion of at least one of said plurality of compartments.

133. The apparatus of claim 88, wherein said cover is configured to receive a plurality of warmers and a plurality of containers.

134. The apparatus of claim 1, wherein said sidewall portion in at least one of said plurality of compartments comprises an interior wall and an exterior wall defining a hollow space therebetween for receiving ornamentation.

135. The apparatus of claim 88, wherein said cover comprises winding means for winding an electrical cord.

136. The apparatus of claim 88, wherein said cover comprises a base and a lid, and wherein at least one of said base and said lid comprises at least one opening for allowing light to pass therethrough.
